(19) United States
(12) Patent Application Publication
(10) Pub. No.: US 2009/0048285 A1
(43) Pub. Date: Feb. 19, 2009

(54) Pyrrolopyridines Useful in the Treatment of Inflammation

(76) Inventors: Benjamin Pelcman, Solna (SE); Kristofer Olofsson, Solna (SE); Pavels Arsenjans, Riga (LV); Ivars Kalvins, Riga (LV); Edgars Suna, Riga (LV); Martins Katkevics, Riga (LV); Marina Madre, Riga (LV); Vita Ozola, Riga (LV)

Correspondence Address: MORGAN LEWIS & BOCKIUS LLP, 1111 PENNSYLVANIA AVENUE NW, WASHINGTON, DC, 20004, US

(21) Appl. No.: 11/795,573

(22) PCT Filed: Jan. 19, 2006
(86) PCT No.: PCT/GB06/00168

§ 371 Date: Mar. 25, 2008

Related U.S. Application Data
(60) Provisional application No. 60/644,526, filed on Jan. 19, 2005.
(60) Provisional application No. 60/644,555, filed on Jan. 19, 2005.
(60) Provisional application No. 60/644,556, filed on Jan. 19, 2005.
(60) Provisional application No. 60/644,557, filed on Jan. 19, 2005.

(51) Int. Cl. A61K 31/437; C07D 471/04; A61P 25/00; A61P 37/00; A61P 31/00

(52) U.S. Cl. 514/300; 546/113

(57) ABSTRACT

There is provided compounds of formula (I), wherein X¹, R¹, R², Y₁, Y₂, Y₃ and Y₄ have meanings given in the description, and pharmaceutically-acceptable salts thereof, which compounds are useful in the treatment of diseases in which inhibition of the activity of a member of the MAPEG family is desired and/or required, and particularly in the treatment of inflammation.

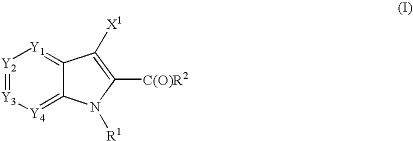

### FIELD OF THE INVENTION

This invention relates to novel pharmaceutically-useful compounds, which compounds are useful as inhibitors of enzymes belonging to the membrane-associated proteins in the eicosanoid and glutathione metabolism (MAPEG) family. Members of the MAPEG family include the microsomal prostaglandin E synthase-1 (mPGES-1), 5-lipoxygenase-activating protein (FLAP), leukotriene C₄ synthase and microsomal glutathione S-transferases (MGST1, MGST2 and MGST3). The compounds are of potential utility in the treatment of inflammatory diseases including respiratory diseases. The invention also relates to the use of such compounds as medicaments, to pharmaceutical compositions containing them, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Inflammatory diseases that affect the population include asthma, inflammatory bowel disease, rheumatoid arthritis, osteoarthritis, rhinitis, conjunctivitis and dermatitis.

Inflammation is also a common cause of pain. Inflammatory pain may arise for numerous reasons, such as infection, surgery or other trauma. Moreover, several diseases including malignancies and cardiovascular diseases are known to have inflammatory components adding to the symptomotology of the patients.

Asthma is a disease of the airways that contains elements of both inflammation and bronchoconstriction. Treatment regimens for asthma are based on the severity of the condition. Mild cases are either untreated or are only treated with inhaled β-agonists which affect the bronchoconstriction element, whereas patients with more severe asthma typically are treated regularly with inhaled corticosteroids which to a large extent are anti-inflammatory in their nature.

Another common disease of the airways with inflammatory and bronchoconstrictive components is chronic obstructive pulmonary disease (COPD). The disease is potentially lethal, and the morbidity and mortality from the condition is considerable. At present, there is no known pharmacological treatment capable of changing the course of the disease.

The cyclooxygenase (COX) enzyme exists in two forms, one that is constitutively expressed in many cells and tissues (COX-1), and one that is induced by pro-inflammatory stimuli, such as cytokines, during an inflammatory response (COX-2).

COXs metabolise arachidonic acid to the unstable intermediate prostaglandin H₂ (PGH₂). PGH₂ is further metabolized to other prostaglandins including PGE₂, PGF_{2α}, PGD₂, prostacyclin and thromboxane A₂. These arachidonic acid metabolites are known to have pronounced physiological and pathophysiological activity including pro-inflammatory effects.

PGE₂ in particular is known to be a strong pro-inflammatory mediator, and is also known to induce fever and pain. Consequently, numerous drugs have been developed with a view to inhibiting the formation of PGE₂, including “NSAIDs” (non-steroidal antiinflammatory drugs) and “coxibs” (selective COX-2 inhibitors). These drugs act predominantly by inhibition of COX-1 and/or COX-2, thereby reducing the formation of PGE₂.

However, the inhibition of COXs has the disadvantage that it results in the reduction of the formation of all metabolites of arachidonic acid, some of which are known to have beneficial properties. In view of this, drugs which act by inhibition of COXs are therefore known/suspected to cause adverse biological effects. For example, the non-selective inhibition of COXs by NSAIDs may give rise to gastrointestinal side-effects and affect platelet and renal function. Even the selective inhibition of COX-2 by coxibs, whilst reducing such gastrointestinal side-effects, is believed to give rise to cardiovascular problems.

An alternative treatment of inflammatory diseases that does not give rise to the above-mentioned side effects would thus be of real benefit in the clinic. In particular, a drug that inhibits (preferably selectively) the transformation of PGH₂ to the pro-inflammatory mediator PGE₂ might be expected to reduce the inflammatory response in the absence of a corresponding reduction of the formation of other, beneficial arachidonic acid metabolites. Such inhibition would accordingly be expected to alleviate the undesirable side-effects mentioned above.

PGH₂ may be transformed to PGE₂ by prostaglandin E synthases (PGES). Two microsomal prostaglandin E synthases (mPGES-1 and mPGES-2), and one cytosolic prostaglandin E synthase (cPGES) have been described.

The leukotrienes (LTs) are formed from arachidonic acid by a set of enzymes distinct from those in the COX/PGES pathway. Leukotriene B4 is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. The biological activities of the CysLTs are mediated through two receptors designated CysLT₁ and CysLT₂. As an alternative to steroids, leukotriene receptor antagonists (LTRas) have been developed in the treatment of asthma. These drugs may be given orally, but do not control inflammation satisfactorily. The presently used LTRas are highly selective for CysLT₁. It may be hypothesized that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs. Among these proteins, 5-lipoxygenase, 5-lipoxygenase-activating protein (FLAP), and leukotriene C₄ synthase may be mentioned. A FLAP inhibitor would also decrease the formation of the proinflammatory LTB₄.

mPGES-1, FLAP and leukotriene C₄ synthase belong to the membrane-associated proteins in the eicosanoid and glutathione metabolism (MAPEG) family. Other members of this family include the microsomal glutathione S-transferases (MGST1, MGST2 and MGST3). For a review, c.f. P.-J. Jacobsson et al in Am. J. Respir. Crit. Care Med. 161, S20 (2000). It is well known that compounds prepared as antagonists to one of the MAPEGs may also exhibit inhibitory activity towards other family members, c.f. J. H Hutchinson et al in J Med. Chem. 38, 4538 (1995) and D. Claveau et al in J. Immunol. 170, 4738 (2003). The former paper also describes that such compounds may also display notable cross-reactivity with proteins in the arachidonic acid cascade that do not belong to the MAPEG family, e.g. 5-lipoxygenase.

Thus, agents that are capable of inhibiting the action of mPGES-1, and thus reducing the formation of the specific arachidonic acid metabolite PGE₂, are likely to be of benefit in the treatment of inflammation. Further, agents that are capable of inhibiting the action of the proteins involved in the synthesis of the leukotrienes are also likely to be of benefit in the treatment of asthma and COPD.

### PRIOR ART

Indole-2-carboxylates, and derivatives thereof, are disclosed in international patent applications WO 2005/005415, WO 2005/123675, WO 2005/123673 and WO 2005/123674 for use as inhibitors of mPGES and thus in the treatment of inflammation. International patent application WO 00/46198 also discloses indoles for potential use in the treatment of inflammation. However, pyrrolopyridines are neither mentioned nor suggested in any of these documents.

International patent application WO 95/33748 discloses various pyrrolopyridines for use in the treatment of cardiovascular and renal diseases. Compounds with aryl or heteroaryl groups attached to (a) the pyridine ring, via a linker or otherwise; and/or (b) the 1(N)-position of the pyrrole ring, are not specifically disclosed in this document.

P. J. Roy et al, Synthesis, 16 (2005), 2751-2757, N. Lachance et al, Synthesis, 15 (2005), 2571-2577 and Dropinski et al, Bioorganic and Medicinal Chemistry Letters, 15 (2005), 5035-5038 all discloses various pyrrolopyridines. However, there is no mention or suggestion in any of these documents of compounds in which the pyridine ring of the pyrrolopyridine is substituted with an aromatic group (via a linker or otherwise), nor do these documents suggest the use of such compounds as inhibitors of in PGES.

### DISCLOSURE OF THE INVENTION

According to the invention there is provided a compound of formula I,

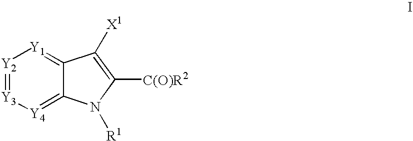

wherein

one of Y₁, T₂, Y₃ and Y₄ represents —N═ and the others respectively represent —C(R³)═, —C(R⁴)═ and —C(R⁵)═;

R² represents —OR^{6a} or —N(R^{6b})R⁷;

X¹ represents H, halo, —N(R⁸)-J-R⁹ or -Q-X²;

J represents a single bond, —C(O)— or —S(O)ₘ—;

Q represents a single bond, —O—, —C(O)— or —S(O)ₘ—;

X² represents:

(a) C₁₋₈ alkyl or a heterocycloalkyl group, both of which are optionally substituted by one or more substituents selected from G¹ and/or Z¹; or

(b) an aryl group or a heteroaryl group, both of which are optionally substituted by one or more substituents selected from A;

one of the groups R³, R⁴ and R⁵ represents -D-E and:

a) the other groups are independently selected from hydrogen, G¹, an aryl group, a heteroaryl group (which latter two groups are optionally substituted by one or more substituents selected from A), C₁₋₈ alkyl and a heterocycloalkyl group (which latter two groups are optionally substituted by one or more substituents selected from G¹ and/or Z¹); and/or

b) any two other groups which are adjacent to each other are optionally linked to form, along with two carbon atoms of the essential pyridine ring in the compound of formula I, a 3- to 8-membered ring, optionally containing 1 to 3 heteroatoms, which ring is itself optionally substituted by one or more substituents selected from halo, —R^{6c}, —OR^{6d} and ═O;

D represents a single bond, —O—, —C(R¹⁰)(R¹¹)—, C₂₋₄ alkylene, —C(O)— or —S(O)ₘ—;

m represents, on each occasion when mentioned above, 0, 1 or 2;

R¹ and E independently represent an aryl group or a heteroaryl group, both of which groups are optionally substituted by one or more substituents selected from A;

R^{6a}, R^{6b}, R^{6c}, R^{6d}, R⁷, R⁸ and R⁹ independently represent, on each occasion when mentioned above:

I) hydrogen;

II) an aryl group or a heteroaryl group, both of which are optionally substituted by one or more substituents selected from B; or

III) C₁₋₈ alkyl or a heterocycloalkyl group, both of which are optionally substituted by one or more substituents selected from G¹ and/or Z¹; or R^{6b} and R⁷, and R⁸ and R⁹ (as appropriate) may be linked together to form, along with the N atom and (in the case of R⁹) the J group to which they are attached, a 3- to 8-membered ring, optionally containing 1 to 3 heteroatoms and/or 1 to 3 double bonds, which ring is optionally substituted by one or more substituents selected from G¹ and/or Z¹;

R¹⁰ and R¹¹ independently represent H, halo or C₁₆ allyl, which latter group is optionally substituted by halo, or R¹⁰ and R¹¹ may together form, along with the carbon atom to which they are attached, a 3- to 6-membered ring, which ring optionally contains a heteroatom and is optionally substituted by one or more substituents selected from halo and C₁₋₃ alkyl, which latter group is optionally substituted by one or more halo substituents;

A represents, on each occasion when mentioned above:

I) an aryl group or a heteroaryl group, both of which are optionally substituted by one or more substituents selected from B;

II) C₁₋₈ alkyl or a heterocycloalkyl group, both of which are optionally substituted by one or more substituents selected from G¹ and/or Z¹; or

III) a G¹ group;

G¹ represents, on each occasion when mentioned above, halo, cyano, —N₃, —NO₂, —ONO₂ or -A¹-R^{12a};

wherein A¹ represents a single bond or a spacer group selected from —C(O)A²-, —S(O)₂A³-, —N(R^{13a})A⁴- or —OA⁵-, in which:

A² represents a single bond, —O—, —N(R^{13b})— or —C(O)—;

A³ represents a single bond, —O— or —N(R^{13c})₅;

A⁴ and A⁵ independently represent a single bond, —C(O)—, —C(O)N(R^{13d})—, —C(O)O—, —S(O)₂— or —S(O)₂N(R^{13e})—;

Z¹ represents, on each occasion when mentioned above, ═O, ═S, ═NOR^{12b}, —NS(O)₂N(R^{13f})R², ═NCN or ═C(H)NO₂;

B represents, on each occasion when mentioned above:

I) an aryl group or a heteroaryl group, both of which are optionally substituted by one or more substituents selected from G²;

II) C₁₋₈ alkyl or a heterocycloalkyl group, both of which are optionally substituted by one or more substituents selected from G² and/or Z²; or

III) a G² group;

G² represents, on each occasion when mentioned above, halo, cyano, —N₃, —NO₂, —ONO₂ or -A⁶-R^{14a};

wherein A⁶ represents a single bond or a spacer group selected from —C(O)A⁷-; —S(O)₂A⁸-, N(R^{15a})A⁹- or —OA¹⁰-, in which:

A⁷ represents a single bond, —O—, —N(R^{15b})— or —C(O)—;

A⁸ represents a single bond, —O— or —N(R^{15c})—;

A⁹ and A¹⁰ independently represent a single bond, —C(O)—, —C(O)N(R^{15d})—, —C(O)O—, —S(O)₂— or —S(O)₂N(R^{15e})—;

Z² represents, on each occasion when mentioned above, ═O, ═S, ═NOR^{14b}, ═NS(O)₂N(R^{15f})R^{14c}, ═NCN or ═C(H)NO₂;

R^{12a}, R^{12b}, R^{12c}, R^{13a}, R^{13b}, R^{13c}, R^{13d}, R^{13e}, R^{13f}, R^{14a}, R^{14b}, R^{14c}, R^{15a}, R^{15b}, R^{15c}, R^{15d}, R^{15e} and R^{15f} are independently selected from:

i) hydrogen;

ii) an aryl group or a heteroaryl group, both of which are optionally substituted by one or more substituents selected from G³;

iii) C₁₋₈ alkyl or a heterocycloalkyl group, both of which are optionally substituted by one or more substituents selected from G³ and/or Z³; or any pair of R^{12a} to R^{12c} and R^{13a} to R^{13f}, and/or R^{14a} to R^{14c} and R^{15a} to R^{15f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;

G³ represents, on each occasion when mentioned above, halo, cyano, —N₃, —NO₂, —ONO₂ or -A¹¹-R^{16a};

wherein A¹¹ represents a single bond or a spacer group selected from —C(O)A¹²-, —S(O)₂A¹³-, —N(R^{17a})A¹⁴- or —OA¹⁵-, in which:

A¹² represents a single bond, —O—, —N(R^{17b})— or —C(O)—;

A¹³ represents a single bond, —O— or —N(R^{17c})—;

A¹⁴ and A¹⁵ independently represent a single bond, —C(O)—, —C(O)N(R^{17d})—, —C(O)O—, —S(O)₂— or —S(O)₂N(R^{17e})—;

Z³ represents, on each occasion when mentioned above, ═O, ═S, ═NOR^{16b}, ═NS(O)₂N(R^{17f})R^{16c}, ═NCN or ═C(H)NO₂;

R^{16a}, R^{16b}, R^{16c}, R^{17a}, R^{17b}, R^{17c}, R^{17d}, R^{17e} and R^{17f} are independently selected from:

i) hydrogen;

ii) C₁₋₆ alkyl or a heterocycloalkyl group, both of which groups are optionally substituted by one or more substituents selected from halo, C₁₋₄ alkyl, —N(R^{18a})R^{19a}, —OR^{18b} and ═O; and

iii) an aryl or heteroaryl group, both of which are optionally substituted by one or more substituents selected from halo, Cl₄ allyl, —N(RISC)R^{19b} and —OR^{18d}; or any pair of R^{16a} to R^{16c} and R^{17a} to R^{17f} may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from halo, C₁₋₄ allyl, —N(R^{18e})R^{19c}, —OR^{18f} and ═O;

R^{18a}, R^{18b}, R^{18c}, R^{18d}, R^{18e}, R^{18f}, R^{19a}, R^{19b} and R^{19c} are independently selected from hydrogen and C₁₋₄ alkyl, which latter group is optionally substituted by one or more halo groups;

or a pharmaceutically-acceptable salt thereof,

which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism.

Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl, and C_{1-q} alkylene, groups (where q is the upper limit of the range) defined herein may be straight-chain or, when there is a sufficient number (i.e. a minimum of two or three, as appropriate) of carbon atoms, be branched-chain, and/or cyclic (so forming, in the case of alkyl, a C_{3-q} cycloalkyl group). Further, when there is a sufficient number (i.e. a minimum of four) of carbon atoms, such groups may also be part cyclic. Such alkyl and alkylene groups may also be saturated or, when there is a sufficient number (i.e. a minimum of two) of carbon atoms, be unsaturated (forming, for example, in the case of alkyl, a C_{2-q} alkenyl or a C_{2-q} allynyl group or, in the case of alkylene, a C₂-q alkenylene or a C_{2-q} alkynylene group).

C_{3-q} cycloalkyl groups (where q is the upper limit of the range) that may be mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double or triple bonds (forming for example a C_{3-q} cycloalkenyl or a C_{8-q} cycloalkynyl group). Substituents may be attached at any point on the cycloalkyl group. Further in the case where the substituent is another cyclic compound, then the cyclic substituent may be attached through a single atom on the cycloalkyl group, forming a so-called “spiro”-compound.

The term “halo”, when used herein, includes fluoro, chloro, bromo and iodo.

Heterocycloalkyl groups that may be mentioned include non-aromatic monocyclic and bicyclic groups heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{8-q} heterocycloalkynyl group. C_{2-q} heterocycloalkyl groups that may be mentioned include 7-azabicyclo[2.2.1]-heptanyl, 6-azabicyclo[3.1.1]heptanyl, 6-azabicyclo[3.2.1]-octanyl, 8-aziridinyl-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo [2.2.1]heptanyl, 6-oxabicyclo-[3.2.1]octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the other substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form.

For the avoidance of doubt, the term “bicyclic”, when employed in the context of cycloalkyl and heterocycloalkyl groups refers to such groups in which the second ring is formed between two adjacent atoms of the first ring. The term “bridged”, when employed in the context of cycloalkyl or heterocycloalkyl groups refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic, bicyclic or tricyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heterocyclic groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S- oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which R¹ and X² are both aryl groups substituted by one or more C₁₋₈ alkyl groups, the alkyl groups in question may be the same or different. Similarly, when groups are substituted by more than one substituent as defined herein, the identities of those individual substituents are not to be regarded as being interdependent. For example, when X² and/or R¹ represents e.g. an aryl group substituted by G¹ in addition to, for example, C₁₋₈ alkyl, which latter group is substituted by G¹, the identities of the two G¹ groups are not to be regarded as being interdependent.

For the avoidance of doubt, in four specific embodiments of the invention, any one of Y₁, Y₂, Y₃ or Y₄ represents —N═ and the others respectively represent —C(R³)═, —C(R⁴)═ and —C(R⁵)═.

The skilled person will appreciate that when one of the R³ to R⁵ groups represents -D-E and any two other adjacent groups are linked to form another ring, then in the case where Y₂, for example, represents —N═, then only R⁴ and R⁵ may be linked.

For the avoidance of doubt, when a term such as “R³ to R⁵” is employed herein, this will be understood by the skilled person to mean R³, R⁴ and R⁵ inclusively.

As stated hereinbefore, any pair of R^{12a}, to R^{12c} and R^{13a} to R^{13f} may be linked as hereinbefore defined. For the avoidance of doubt, such R^{12a} to R^{12c} groups, and R^{13a} to R^{13f} groups may be attached to a single nitrogen atom (e.g. R^{12a}, and R^{13a} or R^{12c} and R^{13f}), which may form part of the ring.

Preferred compounds of the invention include those in which:

A represents G¹ or C₁₋₇ (e.g. C₁₋₆) alkyl optionally substituted by one or more G¹ groups;

X² represents C₁₋₆ alkyl or heterocycloalkyl, both of which are optionally substituted by one or more G¹ and/or Z¹ groups;

R⁸ represents H or C₁₋₂ alkyl (e.g. methyl);

R⁹ represents H or, preferably, C₁₋₆ (e.g. C₁₋₃) alkyl, which alkyl group may be unsubstituted, but is preferably substituted by one or more (e.g. one) groups selected from G¹, or an aryl group optionally substituted by one or more B groups; or R⁸ and R⁹ are linked to form a 4- to 7-membered (such as a 4- to 6- (e.g. 5- or 6-) membered) ring, which ring may, for example preferably, contain (in addition to the nitrogen atom and the J group to which R⁸ and R⁹ are respectively attached) a further heteroatom (e.g. nitrogen or oxygen) and which ring is optionally substituted by one or more (e.g. two) Z¹ groups;

G¹ represents halo, cyano, —NO₂ or -A¹-R^{12a};

A¹ represents a single bond or, preferably (e.g. in the case where Y₄ represents —N═), —C(O)A²-, —N(R^{13a})A⁴- or —OA⁵-;

A² represents —O—;

A⁴ and A⁵ independently represent a single bond, —C(O)—, —C(O)N(R^{13d})— or —C(O)O—;

R^{12a} to R^{12c}, independently represent hydrogen, an aryl group, a heteroaryl group, C₁₋₆ alkyl or a heterocycloalkyl group (such as C₄₋₈ heterocycloalkyl, which group contains one nitrogen atom and, optionally, a further nitrogen or oxygen atom), which latter four groups are optionally substituted by one or more G³ groups and/or (in the case of alkyl and heterocycloalkyl) Z³ groups;

R^{13a} to R^{13f} independently represent C₁₋₂ alkyl or, preferably, hydrogen;

Z¹ represents ═NOR^{12b}, ═NCN or, preferably, ═O;

R^{6a} to R^{6d} and R⁷ independently represent H or C₁₋₃ alkyl optionally substituted by one or more halo (e.g. fluoro) groups;

G² represents halo, —NO₂ or -A -R^{14a};

A⁶ represents —N(R^{15a})A⁹- or —OA¹⁰-;

A⁹ represents —C(O)N(R^{15d})—, —C(O)O— or, more preferably, a single bond or —C(O)—;

A¹⁰ represents a single bond;

Z² represents ═NOR^{14b}, ═NCN or, more preferably, ═O;

G³ represents halo, —NO₂ or -A¹¹-R^{16a};

A¹¹ represents a single bond or, more preferably, —N(R^{17a})— or —O—;

R^{16a} to R^{16c} independently represent an optionally substituted C₁₋₆ alkyl or aryl group;

Z³ represents ═O;

J represents a single bond, —C(O)— or —S(O)₂—;

when any one of R^{16a}, R^{16b}, R^{16c}, R^{17a}, R^{17b}, R^{17c}, R^{17d}, R^{17e} and R^{17f} represents optionally substituted C₁₋₆ alkyl, the optional substituent is one or more halo groups;

when any one of R^{18a}, R^{18b}, R^{18c}, R^{18d}, R^{18e}, R^{18f}, R^{19a}, R^{19b} and R^{19c} represents optionally substituted C₂— allyl, the optional substituent is one or more fluoro groups.

Preferred aryl and heteroaryl groups that R¹, X² (when X² represents an aryl or heteroaryl group) and E may represent include optionally substituted phenyl, naphthyl, pyrrolyl, furanyl, thienyl, pyrazolyl, imidazolyl (e.g 1-imidazolyl, 2-imidazolyl or 4-imidazolyl), oxazolyl, isoxazolyl, thiazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, groups.

Preferred values of R¹ and E include optionally substituted pyridyl (e.g. 2-pyridyl), phenyl or imidazolyl.

Optional substituents on R¹, X² (when X² represents an aryl or heteroaryl group) and E groups are preferably selected from:

halo (e.g. fluoro, bromo or, preferably, chloro);

cyano;

—NO₂;

C₁₋₆ alkyl, which alkyl group may be linear or branched (e.g. C₁₋₄ alkyl (including ethyl, n-propyl, isopropyl, n-butyl or, preferably, methyl or t-butyl), n-pentyl, isopentyl, n-hexyl or isohexyl), cyclic (e.g. cyclopropyl, cyclobutyl, cyclopentyl or cyclohexyl), part-cyclic (e.g. cyclopropylmethyl), unsaturated (e.g. 1-propenyl, 2-propenyl, 1-butenyl, 2-butenyl, 3-butenyl, 1-pentenyl, 2-pentenyl, 4-pentenyl or 5-hexenyl) and/or optionally substituted with one or more halo (e.g. fluoro) group (so forming, for example, fluoromethyl, difluoromethyl or, preferably, trifluoromethyl);

heterocycloalkyl, such as a C₄₋₅ heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O;

—OR²⁰; and

—N(R²⁰)R²¹;

wherein R²⁰ and R²¹ independently represent, on each occasion when mentioned above, H or linear, branched or cyclic C₁₋₆ alkyl, such as methyl, ethyl, n-propyl, isopropyl, cyclopropyl, n-butyl, t-butyl, cyclobutyl, cyclopentyl or cyclohexyl (which alkyl groups are optionally substituted by one or more halo (e.g. fluoro) groups (to form e.g. a trifluoromethyl group)). Preferred values of such C₁₋₆ alkyl groups when Y₁ represents —N═ include methyl and isopropyl, when Y₂ or Y₄ represent —N═, isopropyl and cyclopropyl and when Y₃ represents —N═, isopropyl.

Preferred values of R² include —OR^{6a}. Preferred values of R^{6a}, R^{6b}, R^{6c} and R^{6d} include H.

Preferably (and particularly so when Y₂ or Y₃ represent —N═), when X¹ represents:

-Q-X², then Q is a single bond;

-Q-X², then X² represents C₁₋₄ allyl (e.g. isopropyl or, preferably, methyl or ethyl) optionally substituted by one or more G¹ groups.

Preferably (when X¹ represents —N(R⁸)-J-R⁹, then):

J represents —C(O)—;

R⁸ represents H; and/or

R⁹ represents H, C₁₋₅ allyl (e.g. butyl), optionally substituted by one or more G¹ groups, or a phenyl group, optionally substituted by one or more B groups; or R⁸ and R⁹ are linked together to form a propylene or a butylene chain to form, together with the nitrogen atom and the J group to which they are respectively attached, a 5- or 6-membered ring, such as an optionally substituted pyrrolidin-1-yl ring, e.g. a pyrrolidinon-1-yl ring.

Preferably,

B represents G²;

G² represents halo (e.g. chloro).

More preferred compounds include those in which:

one of R³ and R⁴ represents -D-E and the other represents aryl (e.g. phenyl) optionally substituted by one or more A groups, or, more preferably, H or, for example in the case where Y₂ represents —N═, G¹. In the case where Y₄ or Y₃ represent —N═, R⁴ more preferably represents -D-E and R³ more preferably represents H. In the case where Y₁ represents —N═, R³ more preferably represents -D-E and R⁴ more preferably represents H;

D represents —O— or, more preferably (e.g. in the case when Y₂ or Y₄ represent —N═), a single bond;

R⁵ represents H;

A represents G¹ or C₁₋₆ (e.g. C₁₋₅) alkyl (e.g. t-butyl or, more preferably, methyl or (e.g. in the case when Y₂ or Y₄ represent —N═) cyclohexyl) optionally substituted by one or more G¹ groups;

X¹ represents H, halo (e.g. chloro or fluoro) or -Q-X². It is more preferred that when Y₃ represents —N═, then X¹ represents H or halo (e.g. chloro or fluoro) and when Y₂ represents —N═, then X¹ represents -Q-X² or, more preferably, H;

Q represents (e.g. in the case where Y₁ or Y₄ represents —N═)—S— or, e.g. preferably in the case where Y₄ represents —N═, —O— or, preferably, a single bond;

X² represents C₁₋₄ alkyl (e.g. isopropyl or, for example preferably in the case where Y₁ represents —N═, methyl or ethyl) optionally substituted by one or more G¹ groups;

G¹ represents cyano or, more preferably (e.g. in the case when Y₂, Y₃ or Y₄ represent —N═), halo (e.g. chloro or fluoro) or -A¹-R^{12a};

A⁴ and A⁵ independently represent a single bond;

R^{12a} to R^{12c} independently represent a phenyl group, a heteroaryl (such as tetrazolyl (e.g. 5-tetrazolyl), imidazolyl (e.g. 4-imidazolyl or 2-imidazolyl) or pyridyl (e.g. 3-pyridyl, 4-pyridyl or, especially, 2-pyridyl) group, both of which groups are optionally substituted by one or more G³ groups or, more preferably, H or a C₁₋₄ alkyl (e.g. methyl or, more preferably isopropyl or cyclopropyl) group, which latter group is optionally substituted by one or more G³ groups;

G³ represents -A¹¹-R^{16a} or, more preferably, halo (e.g. fluoro);

A¹¹ represents a single bond;

R^{16a} to R^{16c} independently represent C₁₋₂ alkyl (e.g. methyl) optionally substituted by one or more fluoro atoms.

More preferred (particularly in the case where Y₃, Y₂ or Y₁, represent —N═) compounds include those in which:

when X¹ represents halo, it represents chloro or fluoro;

A¹ represents a single bond or, more preferably in the case where Y₂ or Y₃ represent —N═, —OA⁵- or, e.g. in the case where Y₂ represents —N═, —N(R^{13a})A⁴-;

R^{12a} to R^{12c} independently represent a phenyl group, a heteroaryl group as hereinbefore defined, or, more preferably, a C₁₋₄ alkyl (e.g. methyl, more preferably (for example in the case where Y₃ represents —N═), isopropyl or, e.g. in the case where Y₂ represents —N═, cyclopropyl) group, all of which are optionally substituted by one or more G³ groups.

Values of R¹ that may be mentioned include 4-cyclopropoxyphenyl, 4-cyclopentyloxphenyl and 4-isopropoxyphenyl.

Values of E that may be mentioned include 3-trifluoromethylphenyl or, more preferably, 5-trifluoromethylpyrid-2-yl, 4-cyclohexylphenyl, 3-chlorophenyl, and 4-trifluoromethylphenyl. 5-Trifluoromethylpyrid-2-yl and 4-cyclohexylphenyl are particularly preferred when D represents a single bond in the case when any one of Y₂, Y₃ or Y₄ represents —N═. 3-Chlorophenyl is particularly preferred when D represents —O—. 5-Trifluoromethylpyrid-2-yl and 4-cyclohexylphenyl are particularly preferred when D represents —O— in the case when Y₁ is —N═. 4-Trifluoromethylphenyl is particularly preferred when D represents a single bond.

Particularly preferred values of X² include C₁₋₃ alkyl (e.g. methyl), which group is unsubstituted or, preferably, substituted by one or more halo (e.g. fluoro or chloro) groups so forming, for example, a trifluoromethyl group.

Compounds of the invention may be made in accordance with techniques that are well known to those skilled in the art, for example as described hereinafter.

According to a further aspect of the invention there is provided a process for the preparation of a compound of formula I, which process comprises:

(i) reaction of a compound of formula II,

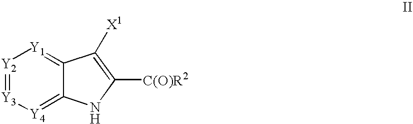

wherein X¹, R², Y₁, Y₂, Y₃ and Y₄ are as hereinbefore defined, with a compound of formula III,

R¹L¹ III

wherein L¹ represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ and R¹ is as hereinbefore defined, for example optionally in the presence of an appropriate metal catalyst (or a salt or complex thereof) such as Cu, Cu(OAc)₂, CuI (or CuI/diamine complex), Pd(OAc)₂, Pd₂(dba)₃ or NiCl₂ and an optional additive such as PPh₃, 2,2′-bis(diphenylphosphino)-1,1′-binaphthyl, xantphos, NaI or an appropriate crown ether, such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof), in a suitable solvent (e.g. dichloromethane, dioxane, toluene, isopropanol, ethanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when R¹ represents phenyl and L¹ represents bromo, i.e. bromobenzene). This reaction may be carried out at room temperature or above (e.g. at a high temperature, such as the reflux temperature of the solvent system that is employed) or using microwave irradiation;

(ii) for compounds of formula I in which X¹ represents -Q-X², in which Q is a single bond or —C(O)—, reaction of a compound of formula IV,

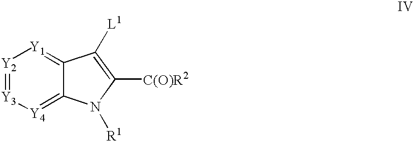

wherein L¹, R¹, R², Y₁, Y₂, Y₃ and Y₄ are as hereinbefore defined, with a compound of formula V,

X²-Q^{a}-L² V

wherein Q^{a} represents a single bond or —C(O)—, L² represents a suitable leaving group such as chloro, bromo, iodo, —B(OH)₂ or a protected derivative thereof, for example a 4,4,5,5-tetramethyl-1,3,2-dioxaborolan-2-yl group, 9-borabicyclo[3.3.1]nonane (9-BBN), —Sn(alkyl)₃ (e.g.—SnMe₃ or —SnBu₃), or a similar group known to the skilled person, and X² is as hereinbefore defined. The skilled person will appreciate that L¹ and L² will be mutually compatible. In this respect, preferred leaving groups for compounds of formula V in which Q^{a} is —C(O)— include chloro or bromo groups, and preferred leaving groups for compounds of formula V in which Q^{a} is a single bond include —B(OH)₂, 4,4,5,5-tetramethyl-1,3,2-dioxaborolan-2-yl, 9-borabicyclo[3.3.1]nonane (9-BBN), or —Sn(alkyl)₃. This reaction may be performed, for example in the presence of a suitable catalyst system, e.g. a metal (or a salt or complex thereof such as CuI, Pd/C, PdCl₂, Pd(OAc)₂, Pd(PPh₃)₂Cl₂, Pd(PPh₃)₄, Pd₂(dba)₃ or NiCl₂ and a ligand such as t-Bu₃P, (C₆H₁₁)₃P, PPh₃, AsPh₃, P(o-Tol)₃, 1,2-bis(diphenylphosphino)-ethane, 2,2′-bis(di-tert-butylphosphino)-1,1′-biphenyl, 2,2′-bis(diphenyl-phosphino)-1,1′-binaphthyl, 1,1′-bis(diphenyl-phosphinoferrocene), 1,3-bis(diphenylphosphino)propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above (e.g. at a high temperature such as the reflux temperature of the solvent system) or using microwave irradiation. The skilled person will appreciate that certain compounds of formula IV (in particular those in which L¹ represents chloro, bromo or iodo) are also compounds of formula I and therefore compounds of the invention. In the case where Q^{a} represents a single bond and X² represents either C₂₋₈ alkenyl, cycloalkenyl or heterocycloalkenyl in which the double bond is between the carbon atoms that are α and β to L², the skilled person will appreciate that the double bond may migrate on formation of the compound of formula I to form a double bond that is between the carbon atoms that are β and γ to the indole ring;

(iii) for compounds of formula I in which X¹ represents -Q-X² and Q represents —C(O)—, reaction of a compound of formula I in which X¹ represents H with a compound of formula V in which Q^{a} represents —C(O)— and L² represents a suitable leaving group such as chloro or bromo, —N(C₁₋₆ alkyl)₂ (e.g. —N(CH₃)₂) or a carboxylate group such as —O—C(O)—X^{2y} in which X^{2y} represents X² or H. In the latter case, X^{2y} and X² are preferably the same, or X^{2y} represents e.g. H, CH₃ or CF₃. This reaction may be performed under suitable conditions known to those skilled in the art, for example in the presence of a suitable Lewis acid (e.g. AlCl₃ or FeCl₃). Reaction of a compound of formula V in which L² represents —N(C₁₋₆ alkyl)₂ and X² represents optionally substituted aryl (e.g. phenyl) or heteroaryl may be performed in the presence of a reagent such as POCl₃, for example under reaction conditions described in Bioorg. Med. Chem. Lett., 14, 4741-4745 (2004). The skilled person will appreciate that in the latter instance, POCl₃ may convert the compound of formula V into one in which L² represents chloro and/or Q^{a} represents a derivative of —C(O)— (e.g. an iminium derivative), which group may be transformed back to a —C(O)— group before or after reaction with the compound of formula I in which X¹ represents H;

(iv) for compounds of formula I in which X¹ represents —N(R⁸)-J-R⁹ or -Q-X² in which Q represents —O— or —S—, reaction of a compound of formula IV as hereinbefore defined with a compound of formula VI,

X^{1b}H VI

in which X^{1b} represents —N(R⁸)-J-R⁹ or -Q-X² and Q represents —O— or —S— and R⁸, J, R⁹ and X² are as hereinbefore defined, for example under reaction conditions as hereinbefore described in respect of either process (i) or (ii) above;

(v) for compounds of formula I in which X¹ represents -Q-X² and Q represents —S—, reaction of a compound of formula I in which X¹ represents H, with a compound of formula VI in which X^{1b} represents -Q-X², Q represents —S— and X² is as hereinbefore defined, for example in the presence of N-chlorosuccinimide and a suitable solvent (e.g. dichloromethane), e.g. as described in infer alia Org. Lett., 819-821 (2004). Alternatively, reaction of a compound of formula VI in which X^{1b} represents -Q-X², Q represents —S— and X² represents an optionally substituted aryl (phenyl) or heteroaryl (e.g. 2-pyridyl) group, may be performed in the presence of PIFA (PhI(OC(O)CF₃)₂) in a suitable solvent such as (CF₃)₂CHOH. Introduction of such an —S—X² group is described in inter alia Bioorg. Med. Chem. Lett., 14, 4741-4745 (2004);

(vi) for compounds of formula I in which X¹ represents -Q-X² and Q represents —S(O)— or —S(O)₂—, oxidation of a corresponding compound of formula I in which Q represents —S— under appropriate oxidation conditions, which will be known to those skilled in the art;

(vii) for compounds of formula I in which X¹ represents -Q-X², X² represents C₁₋₈ alkyl substituted by G¹, G¹ represents -A¹-R^{12a}, A¹ represents —N(R^{13a})A⁴- and A⁴ is a single bond (provided that Q represents a single bond when X² represents substituted C₁, alkyl), reaction of a compound of formula VII,

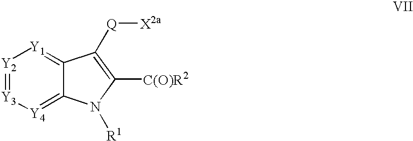

wherein X^{2a} represents a C₁₋₈ alkyl group substituted by a Z¹ group in which Z¹ represents ═O, Q is as hereinbefore defined, provided that it represents a single bond when X^{2a} represents C₁ alkyl substituted by ═O (i.e. —CHO), and R¹, R², Y₁, Y₂, Y₃ and Y₄ are as hereinbefore defined under reductive amination conditions in the presence of a compound of formula VIII,

R^{12a}(R^{13a})NH VIII

wherein R^{12a} and R^{13a} are as hereinbefore defined, under conditions well known to those skilled in the art;

(viia) for compounds of formula I in which X¹ represents -Q-X², Q represents a single bond, X² represents methyl substituted by G¹, G¹ represents -A¹-R^{12a}, A¹ represents —N(R^{13a})A⁴-, A⁴ is a single bond and R^{12a} and R^{13a} are preferably methyl, reaction of a corresponding compound of formula I in which X¹ represents H, with a mixture of formaldehyde (or equivalent reagent) and a compound of formula VIII as hereinbefore defined (e.g. in which R^{12a} and R^{13a} represent methyl), for example in the presence of solvent such as a mixture of acetic acid and water, under e.g. standard Mannich reaction conditions known to those skilled the art;

(viii) for compounds of formula I in which X¹ represents -Q-X², Q represents a single bond and X² represents optionally substituted C₂₋₈ alkenyl (in which a point of unsaturation is between the carbon atoms that are α and β to the indole ring and the optional substituents are preferably other than G¹ in which G¹ represents -A¹-R^{12a}, A¹ represents —OA⁵- or —N(R^{13a})A⁴-, A⁴ and A⁵ both represent a single bond and R^{12a} represents hydrogen), reaction of a corresponding compound of formula IV in which L¹ represents halo (e.g. iodo) with a compound of formula IXA,

H₂C═C(H)X^{2b} IXA

or, depending upon the geometry of the double bond, reaction of a compound of formula VII in which Q represents a single bond and X^{2a} represents —CHO with either a compound of formula IXB,

(EtO)₂P(O)CH₂X^{2b} IXB

or the like, or a compound of formula IXC,

(Ph)₃P═CHX^{2b} IXC

or the like, wherein, in each case, X^{2b} represents H, G¹ (wherein G¹ is preferably other than -A¹-R^{12a}, in which A¹ represents —OA⁵- or —N(R^{13a})A⁴-, A⁴ and A⁵ both represent a single bond and R^{12a} represents hydrogen) or C₁₋₆ alkyl optionally substituted with one of more substituents selected from G¹ and/or Z¹ and G¹ and Z¹ are as hereinbefore defined, for example, in the case of a reaction of a compound of formula IV with compound of formula IXA, in the presence of an appropriate catalyst (such as PdCl₂(PPh₃)₂), a suitable base (e.g. NaOAc and/or triethylamine) and an organic solvent (e.g. DMF) and, in the case of reaction of a compound of formula VII with either a compound of formula IXB, or IXC, under standard Horner-Wadsworth-Emmons, or Wittig, reaction conditions, respectively;

(ix) for compounds of formula I in which X¹ represents -Q-X² and X² represents optionally substituted, saturated C₂₋₈ allyl, saturated cycloalkyl, saturated heterocycloalkyl, C₂₋₈ alkenyl, cycloalkenyl or heterocycloalkenyl, reduction (e.g. hydrogenation) of a corresponding compound of formula I in which X² represents optionally substituted C₂₋₈ alkenyl, cycloalkenyl, heterocycloalkenyl, C₂₋₈ alkynyl, cycloalkynyl or heterocycloalkynyl (as appropriate) under conditions that are known to those skilled in the art. For example, in the case where an alkynyl group is converted to an alkenyl group, in the presence of an appropriate poisoned catalyst (e.g. Lindlar's catalyst);

(x) for compounds of formula I in which D represents a single bond, —C(O)—, —C(R¹⁰)(R¹¹)—, C₂₋₄ alkylene or —S(O)₂—, reaction of a compound of formula X,

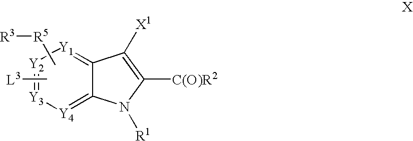

wherein L³ represents L¹ or L² as hereinbefore defined, which group is attached to one or more of the carbon atoms of the pyridine ring of the pyrrolopyridine (i.e. one of the carbon atoms of —C(R³)═, —C(R⁴)═ and —C(R⁵)═, and in place of the R³, R⁴ or R⁵ substituents, as appropriate), R³-R⁵ represents whichever of the two other substituents on the pyridine ring, i.e. R³, R⁴ and R⁵, are already present in that ring, and X¹, R¹, R², Y₁ to Y₄, R³, R⁴ and R⁵ are as hereinbefore defined, with a compound of formula XI,

E-D^{a}-L⁴ XI

wherein D^{a} represents a single bond, —C(O)—, —C(R¹⁰)(R¹¹)—, C₂₋₄ allylene or —S(O)₂—, L⁴ represents L¹ (when L³ is L²) or L² (when L³ is L¹) and L¹, L², E, R¹⁰ and R¹¹ are as hereinbefore defined. For example, when D^{a} represents a single bond, —C(O)— or C₂₋₄ alkylene, the reaction may be performed for example under similar conditions to those described hereinbefore in respect of process step (ii) above. Further, when D^{a} represents —C(O)—, —C(R¹⁰)(R¹¹)—, C₂₋₄ alkylene or —S(O)₂—, the reaction may be performed by first activating the compound of formula X. The skilled person will appreciate that compounds of formula X may be activated when L³ represents halo, by:
(I) forming the corresponding Grignard reagent under standard conditions known to those skilled in the art (e.g. employing magnesium or a suitable reagent such as a mixture of C₁₋₆ alkyl-Mg-halide and ZnCl₂ or LiCl), followed by reaction with a compound of formula XI, optionally in the presence of a catalyst (e.g. FeCl₃) under conditions known to those skilled in the art; or
(II) forming the corresponding lithiated compound under halogen-lithium exchange reaction conditions known to those skilled in the art (e.g. employing n-BuLi or t-BuLi in the presence of a suitable solvent (e.g. a polar aprotic solvent such as THF)), followed by reaction with a compound of formula XI.

The skilled person will also appreciate that the magnesium of the Grignard reagent or the lithium of the lithiated species may be exchanged to a different metal (i.e. a transmetallation reaction may be performed), for example to zinc (e.g. using ZnCl₂) and the intermediate so formed may then be subjected to reaction with a compound of formula XI under conditions known to those skilled in the art, for example such as those described hereinbefore in respect of process (ii) above;

(xi) for compounds of formula I in which D represents —S—, —O— or C₂₋₄ alkynylene in which the triple bond is adjacent to E, reaction of a compound of formula X as hereinbefore defined in which L³ represents L² as hereinbefore defined (for example —B(OH)₂) with a compound of formula XII,

E-D^{b}-H XII

wherein D^{b} represents —S—, —O— or C₂₋₄ alkynylene in which the triple bond is adjacent to E and E is as hereinbefore defined. Such reactions may, be performed under similar conditions to those described hereinbefore in respect of process step (ii) above, for example in the presence of a suitable catalyst system, such as Cu(OAc)₂, a suitable base, such as triethylamine or pyridine, and an appropriate organic solvent, such as DMF or dichloromethane;

(xii) for compounds of formula I in which D represents —S(O)— or —S(O)₂—, oxidation of a corresponding compound of formula I in which D represents —S— under appropriate oxidation conditions, which will be known to those skilled in the art;

(xiii) for compounds of formula I in which D represents —O— or —S—, reaction of a compound of formula XIII,

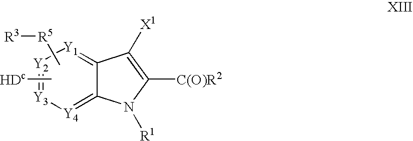

wherein the -Dc^{c}-H group is attached to one or more of the carbon atoms of the pyridine ring of the pyrrolopyridine, DC represents —O— or —S— and X¹, R¹, R², Y₁ to Y₄ and R³-R⁵ are as hereinbefore defined, with a compound of formula XIV,

E-L² XIV

wherein L² is as hereinbefore defined (for example —B(OH)₂, chloro, bromo or iodo) and E is as hereinbefore defined, for example under conditions such as those described hereinbefore in respect of process step (ii) above;

(xiv) for compounds of formula I in which X¹ represents —N(R⁸)-J-R⁹, reaction of a compound of formula XV,

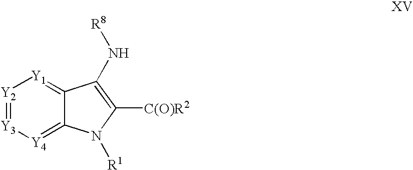

wherein R¹, R², Y₁ to Y₄ and R⁸ are as hereinbefore defined, with a compound of formula XVI,

R⁹-J-L¹ XVI

wherein J, R⁹ and L¹ are as hereinbefore defined, for example at around room temperature or above (e.g. up to 60-70° C.) in the presence of a suitable base (e.g. pyrrolidinopyridine, pyridine, triethylamine, tributylamine, trimethylamine, dimethylaminopyridine, diisopropylamine, 1,8-diazabicyclo [5.4.0]undec-7-ene, sodium hydroxide, or mixtures thereof) and an appropriate solvent (e.g. pyridine, dichloromethane, chloroform, tetrahydrofuran, dimethylformamide, dimethylsulfoxide, water, triethylamine or mixtures thereof) and, in the case of biphasic reaction conditions, optionally in the presence of a phase transfer catalyst;

(xv) for compounds of formula I in which X¹ represents —N(R⁸)-J-R⁹, J represents a single bond and R⁹ represents a C₁₋₈ alkyl group, reduction of a corresponding compound of formula I, in which J represents —C(O)— and R⁹ represents H or a C₁₋₇ alkyl group, in the presence of a suitable reducing agent. A suitable reducing agent may be an appropriate reagent that reduces the amide group to the amine group in the presence of other functional groups (for example an ester or a carboxylic acid). Suitable reducing agents include borane and other reagents known to the skilled person;

(xvi) for compounds of formula I in which X¹ represents halo, reaction of a compound of formula I wherein Xl¹ represents H, with a reagent or mixture of reagents known to be a source of halo atoms. For example, for Br atoms, N-bromosuccinimide, bromine or 1,2-dibromotetrachloroethane may be employed, for I atoms, iodine, diiodoethane, diiodotetrachloroethane or a mixture of NaI or KI and N-chlorosuccinimide may be employed, for Cl atoms, N-chlorosuccinimide may be employed and for F atoms, 1-(chloromethyl)-4-fluoro-1,4-diazoniabicyclo[2.2.2]octane bis(tetrafluoroborate), 1-fluoropyridinium triflate, xenon difluoride, CF₃OF or perchloryl fluoride may be employed. This reaction may be carried out in a suitable solvent (e.g. acetone, benzene or dioxane) under conditions known to the skilled person;

(xvii) for compounds of formula I in which R² represents —OR^{6a} and R^{6a} is other than H, reaction of a compound of formula XVII,

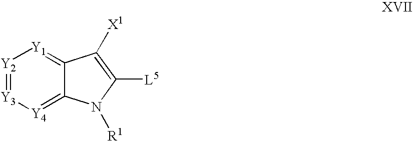

wherein L⁵ represents an appropriate alkali metal group (e.g. sodium, potassium or, especially, lithium), a —Mg-halide, a zinc-based group or a suitable leaving group such as halo or —B(OH)₂, or a protected derivative thereof, and X¹, R¹ and Y₁ to Y₄ are as hereinbefore defined, with a compound of formula XVIII,

L⁶C(O)OR^{6za} XVIII

wherein R^{6za} represents R^{6a} provided that it does not represent H, and L⁶ represents a suitable leaving group such as halo (especially chloro or bromo) under conditions known to those skilled in the art;

(xviii) for compounds of formula I in which R² represents —OR^{6a} and R^{6a} is H, reaction of a compound of formula XVII in which L⁵ represents either:
(I) an alkali metal (for example, such as one defined in respect of process step (xvii) above); or
(II)—Mg-halide,
   with carbon dioxide, followed by acidification under standard conditions known to those skilled in the art, for example, in the presence of aqueous hydrochloric acid;

(xix) for compounds of formula I in which R² represents —OR^{6a}, reaction of a corresponding compound of formula XVII in which L⁵ is a suitable leaving group known to those skilled in the art (such as a sulfonate group (e.g. a triflate) or, preferably, a halo (e.g. bromo or iodo) group) with CO (or a reagent that is a suitable source of CO (e.g. Mo(CO)₆ or CO₂(CO)₈)), in the presence of a compound of formula XIX,

R^{6a}OH XIX

wherein R^{6a} is as hereinbefore defined, and an appropriate catalyst system (e.g. a palladium catalyst such as one described hereinbefore in respect of process step (ii)) under conditions known to those skilled in the art;

(xx) for compounds of formula I in which R² represents —OR⁶ and R^{6a} represents H, hydrolysis of a corresponding compound of formula I in which R^{6a} does not represent H under standard conditions;

(xxi) for compounds of formula I in which R² represents —OR^{6a} and R^{6a} does not represent H:
(A) esterification of a corresponding compound of formula I in which R^{6a} represents H; or
(B) trans-esterification of a corresponding compound of formula I in which R^{6a} does not represent H (and does not represent the same value of R^{6a} as the compound of formula I to be prepared),
   under standard conditions in the presence of the appropriate alcohol of formula XIX as hereinbefore defined but in which R^{6a} represents R^{6za} as hereinbefore defined;

(xxii) for compounds of formula I in which R² represents —N(R^{6b})R⁷, reaction of a corresponding compound of formula I in which R² represents —OR^{6a} with a compound of formula XX,

HN(R^{6b})R⁷ XX

wherein R^{6b} and R⁷ are as hereinbefore defined under standard conditions. For example, the reaction may be performed in the presence of a suitable coupling reagent (e.g. 1,1′-carbonyldiimidazole, N,N′-dicyclohexylcarbodiimide, 1-(3′-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexa-fluorophosphate, benzotriazol-1-yloxytris-pyrrolidinophosphonium hexafluoro-phosphate, bromotrispyrrolidinophosponium hexafluorophosphate, 2-(1H-benzotriazol-1-yl)-1,1,3,3-tetramethyluronium tetrafluorocarbonate or 1-cyclohexylcarbodiimide-3-propyloxymethyl polystyrene, O-(7-azabenzotriazol 1-yl)-N,N,N′,N′-tetramethyluronium hexafluorophosphate or O-benzotriazol-1-yl-N,N,N′,N′,-tetramethyluronium tetrafluoroborate), and/or a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine, butyllithium (e.g. n-, s- or t-butyllithium) or mixtures thereof) and an appropriate solvent (e.g. tetrahydrofuran, pyridine, toluene, dichloromethane, chloroform, acetonitrile, dimethylformamide, dimethylsulfoxide, water, triethylamine or mixtures thereof). Alternatively an azodicarboxylate may be employed under Mitsunobo conditions known to those skilled in the art. The skilled person will appreciate that it may be convenient or necessary to first convert the acid or ester compound of formula I to a corresponding acid halide prior to reaction with the compound of formula XX. Such conversions may be performed in the presence of a suitable reagent (e.g. oxalyl chloride, thionyl chloride, etc) optionally in the presence of an appropriate solvent (e.g. dichloromethane, THF, toluene or benzene) and a suitable catalyst (e.g. DMF), resulting in the formation of the respective acyl chloride. The skilled person will appreciate that when compounds of formula XX are liquid in nature, they may serve as both solvent and reactant in this reaction. An alternative way of performing this step, includes the reaction of a compound of formula I in which R² represents —OR^{6a} in which R^{6a} is other than H (e.g. ethyl) with a compound of formula XX, in the presence of, e.g. trimethylaluminium, for example in an inert atmosphere and in the presence of a suitable solvent (e.g. dichloromethane);

(xxiii) for compounds of formula I in which X¹ represents -Q-X² in which Q represents —O—, reaction of a compound of formula XXI,

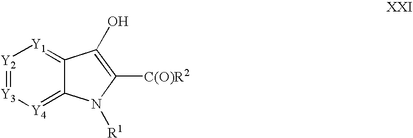

wherein R¹, R² and Y₁ to Y₄ are as hereinbefore defined, with a compound of formula XXII,

X²L⁷ XXII

wherein L⁷ represents a suitable leaving group, such as a halo or sulfonate group and X² is as hereinbefore defined, for example in the presence of a base or under reaction conditions such as those described hereinbefore in respect of process (xiii) above;

(xxiv) for compounds of formula I in which X¹ represents —N(R⁸)-J-R⁹, reaction of a compound of formula XXI as hereinbefore defined, with a compound of formula VI in which X^{1b} represents —N(R⁸)-J-R⁹ and R⁸, R⁹ and J are as hereinbefore defined, for example under reaction conditions known to those skilled in the art (such as those described in Journal of Medicinal Chemistry 1996, Vol. 39, 4044 (e.g. in the presence of MgCl₂));

(xxv) for compounds of formula I in which X¹ represents -Q-X², Q represents a single bond and X² represents C₁₋₈ allyl or heterocycloalkyl substituted u to the indole ring by a G¹ substituent in which G¹ represents -A¹-R^{12a}, A¹ represents —OA⁵-, A⁵ represents a single bond and R^{12a} represents H, reaction of a corresponding compound of formula I in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O, under conditions known to those skilled in the art, for example optionally in the presence of an acid, such as a protic acid or an appropriate Lewis acid. Such substitutions are described in inter alia Bioorg. Med. Chem. Lett., 14, 4741-4745 (2004) and Tetrahedron Lett. 34, 1529 (1993);

(xxvi) for compounds of formula I in which X¹ represents -Q-X², Q represents a single bond and X² represents C₂₋₈ alkyl substituted (e.g. α to the indole ring) by a G¹ substituent in which G¹ represents -A¹-R^{12a}, A¹ represents —OA⁵-, A⁵ represents a single bond and R^{12a} represents H, reaction of a corresponding compound of formula I in which X² represents C₁₋₇ alkyl substituted (e.g. α to the indole ring) by a Z¹ group in which Z¹ represents ═O, with the corresponding Grignard reagent derivative of a compound of formula V in which L² represents chloro, bromo or iodo, Q^{a} is a single bond and X² represents C₁₋₇ alkyl, under conditions known to those skilled in the art;

(xxvii) for compounds of formula I in which X¹ represents -Q-X², Q represents a single bond, and X² represents C₁₋₈ alkyl or heterocycloalkyl, both of which are unsubstituted in the position α to the indole ring, reduction of a corresponding compound of formula I in which X² represents C₁₋₈ alkyl substituted a to the indole ring by a G¹ substituent in which G¹ represents -A¹-R^{12a}, A¹ represents —OA⁵-, A⁵ represents a single bond and R^{12a} represents H, in the presence of a suitable reducing agent such as a mixture of triethyl silane and a protic acid (e.g. CF₃COOH) or a Lewis acid (e.g. (CH₃)₃SiOS(O)₂CF₃) for example under conditions described in inter alia Bioorg. Med. Chem. Lett., 14, 4741-4745 (2004); or

(xxviii) for compounds of formula I in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula I in which X² represents C₁₋₈ alkyl or heterocycloalkyl, which groups are substituted by one or more Z¹ groups in which Z¹ represents ═O under conditions known to those skilled in the art, for example NaBH₄ in the presence of an acid (e.g. CH₃COOH or CF₃COOH), Wolff-Kishner reduction conditions (i.e. by conversion of the carbonyl group to a hydrazone, followed by base induced elimination) or by conversion of the carbonyl to the thioacetal analogue (e.g. by reaction with a dithiane) followed by reduction with e.g. Raney nickel, all under reaction conditions known to those skilled in the art.

Compounds of formula II may be prepared by:
(a) reaction of a compound of formula XXIII,

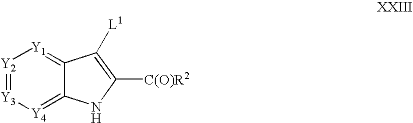

wherein L¹, R² and Y₁ to Y₄ are as hereinbefore defined, with, for compounds of formula II in which X¹ represents:
(1)-Q-X² and Q represents a single bond or —C(O)—, a compound of formula V as hereinbefore defined; or
(2) —N(R⁸)-J-R⁹ or -Q-X², in which Q represents —O— or —S—, a compound of formula VI as hereinbefore defined;
for example under reaction conditions similar to those described hereinbefore in respect of preparation of compounds of formula I (processes (ii) and (iv), respectively) above;
(b) for compounds of formula II in which X¹ represents -Q-X² and Q represents —C(O)—, reaction of a corresponding compound of formula II in which X¹ represents H with a compound of formula V in which Q^{a} represents —C(O)— and L² represents a suitable leaving group, for example under conditions such as those described in respect of preparation of compounds of formula I (process (iii)) above.
(c) for compounds of formula II in which X¹ represents -Q-X² and Q represents —S—, reaction of a corresponding compound of formula II in which X¹ represents H with a compound of formula VI in which X^{1b} represents -Q-X² and Q represents —S—, for example under conditions such as those described hereinbefore in respect of preparation of compounds of formula I (process (v)) above;
(d) for compounds of formula II in which X¹ represents -Q-X² and Q represents —S(O)— or —S(O)₂—, oxidation a corresponding compound of formula II in which Q represent —S—;
(e) for compounds of formula II in which X¹ represents -Q-X², X² represents C₁₋₈ allyl substituted by G¹, G¹ represents -A-R^{12a}, A¹ represents —N(R^{13a})A⁴- and A⁴ is a single bond (provided that Q represents a single bond when X² represents substituted C₁ alkyl), reaction of a compound of formula XXIV,

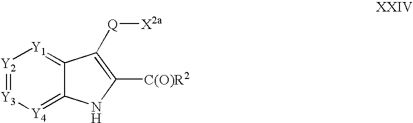

wherein Q, X^{2a}, R² and Y₁ to Y₄ are as hereinbefore defined by reductive amination in the presence of a compound of formula VIII as hereinbefore defined;
(ea) for compounds of formula II in which X¹ represents -Q-X², Q represents a single bond, X² represents methyl substituted by G¹, G¹ represents -A¹-R^{12a}, A¹ represents —N(R^{13a})A⁴-, A⁴ is a single bond and R^{12a} and R^{13a} are preferably methyl, reaction of a corresponding compound of formula II in which X¹ represents H, with a mixture of formaldehyde (or equivalent reagent) and a compound of formula VIII as hereinbefore defined, for example under reaction conditions similar to those described hereinbefore in respect of preparation of compounds of formula I (process (viia)) above;
(f) for compounds of formula II in which X¹ represents -Q-X², Q represents a single bond and X² represents optionally substituted C₂₋₈ alkenyl (in which a point of unsaturation is between the carbon atoms that are α and β to the indole ring and the optional substituents are preferably other than G¹ in which G¹ represents -A¹-R^{12a}, A¹ represents -OA⁵- or —N(R^{13a})A⁴-, A⁴ and A⁵ both represent a single bond and R^{12a} represents hydrogen), reaction of a corresponding compound of formula XXIII in which L¹ represents halo (e.g. iodo) with a compound of formula IXA as hereinbefore defined, or a compound of formula XXIV in which Q represents a single bond and X^{2a} represents —CHO with a compound of formula IXB or a compound of formula IXC as hereinbefore defined, for example under reaction conditions similar to those described hereinbefore in respect of preparation of compounds of formula I (process (viii)) above;
(g) for compounds of formula II in which X¹ represents -Q-X² and X² represents optionally substituted, saturated C₂₋₈ alkyl, saturated cycloalkyl, saturated heterocycloalkyl, C₂-8 alkenyl, cycloalkenyl or heterocycloallcenyl, reduction (e.g. hydrogenation) of a corresponding compound of formula II in which X² represents optionally substituted C₂₋₈ alkenyl, cycloalkenyl, heterocycloalkenyl, C₂₋₈ alkynyl, cycloalkynyl or heterocycloalkynyl (as appropriate);
(h) for compounds of formula II in which D represents a single bond, —C(O)—, —C(R¹⁰)(R¹¹)—, C₂₋₈ alkylene or —S(O)₂—, reaction of a compound of formula XXV,

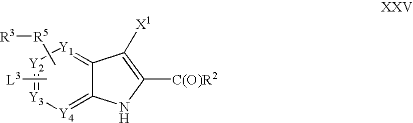

wherein X¹, L³, R², Y₁ to Y₄ and R³-R⁵ are as hereinbefore defined with a compound of formula XI as hereinbefore defined, for example under reaction conditions similar to those described hereinbefore in respect of preparation of compounds of formula I (process (x)) above;
(i) for compounds of formula II in which D represents —S—, —O— or C₂₋₄ alkynylene in which the triple bond is adjacent to E, reaction of a compound of formula XXV as hereinbefore defined in which L³ represents L² as hereinbefore defined (for example —B(OH)₂) with a compound of formula XII as hereinbefore defined, for example under reaction conditions similar to those described hereinbefore in respect of preparation of compounds of formula I (process (xi)) above;
(j) for compounds of formula II in which D represents —S(O)— or —S(O)₂—, oxidation of a corresponding compound of formula II in which D represents —S—;
(k) for compounds of formula II in which D represents —O— or —S—, reaction of a compound of formula XXVI,

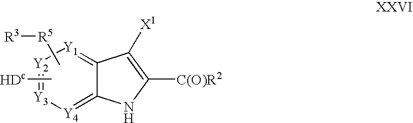

wherein D^{c}, X¹, R², Y₁ to Y₄ and R³-R⁵ are as hereinbefore defined, with a compound of formula XIV as hereinbefore defined;
(l) for compounds of formula II in which X¹ represents —N(R⁸)-J-R⁹, reaction of a compound of formula XXVII,

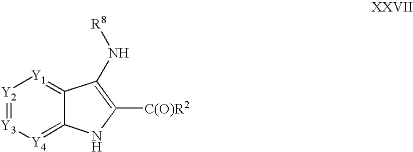

wherein R², Y₁ to Y₄ and R⁸ are as hereinbefore defined with a compound of formula XVI as hereinbefore defined, for example under reaction conditions similar to those described hereinbefore in respect of preparation of compounds of formula I (process (xiv)) above;
(m) for compounds of formula II in which X¹ represents —N(R⁸)-J-R⁹, J represents a single bond and R⁹ represents a C₁₋₈ allyl group, reduction of a corresponding compound of formula II, in which J represents —C(O)— and R⁹ represents H or a C₁₋₇ alkyl group, for example under reaction conditions similar to those described hereinbefore in respect of preparation of compounds of formula I (process (xv)) above;
(n) for compounds of formula II in which X¹ represents halo, reaction of a compound of formula II wherein X¹ represents H, with a reagent or mixture of reagents known to be a source of halo atoms, for example under reaction conditions similar to those described hereinbefore in respect of preparation of compounds of formula I (process (xvi)) above;

(o) for compounds of formula II in which R² represents —OR^{6a} and R^{6a} is other than H, reaction of a compound of formula XXVIII,

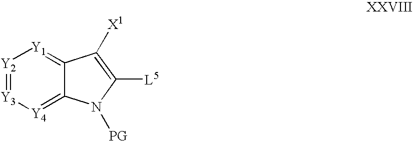

wherein PG represents a suitable protecting group, such as —S(O)₂Ph, —C(O)O⁻, —C(O)OtBu or —C(O)N(Et)₂) and L⁵, X¹ and Y₁ to Y₄ are as hereinbefore defined, with a compound of formula XVIII as hereinbefore defined, or a protected derivative thereof, for example under similar coupling conditions to those described hereinbefore in respect of process (xvii) above, followed by deprotection of the resultant compound under standard conditions;
(p) for compounds of formula II in which R² represents —OR^{6a} and R^{6a} is H, reaction of a compound of formula XXVIII in which L⁵ represents an alkali metal, or —Mg-halide, with carbon dioxide, followed by acidification;
(q) for compounds of formula II in which R² represents —OR^{6a}, reaction of a corresponding compound of formula XXVIII in which L⁵ is a suitable leaving group known to those skilled in the art with CO (or a reagent that is a suitable source of CO), in the presence of a compound of formula XIX as hereinbefore defined;
(r) for compounds of formula II in which R² represents —OR^{6a} and R^{6a} represents H, hydrolysis of a corresponding compound of formula II in which R^{6a} does not represent H;
(s) for compounds of formula II in which R² represents —OR^{6a} and R^{6a} does not represent H:
   (A) esterification of a corresponding compound of formula II in which R^{6a} represents H; or
   (B) trans-esterification of a corresponding compound of formula II in which R^{6a} does not represent H (and does not represent the same value of R^{6a} as the compound of formula II to be prepared);
(t) for compounds of formula II in which R² represents —N(R^{6b})R⁷, reaction of a corresponding compound of formula II in which R² represents —OR^{6a} with a compound of formula XX as hereinbefore defined, for example under reaction conditions similar to those described hereinbefore in respect of preparation of compounds of formula I (process (xxii)) above;
(u) for compounds of formula II in which X¹ represents -Q-X² in which Q represents —O—, reaction of a compound of formula XXIX,

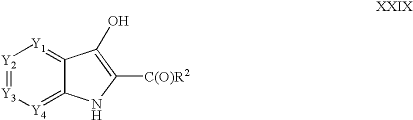

wherein R² and Y₁ to Y₄ are as hereinbefore defined, with a compound of formula XXII as hereinbefore defined, for example under reaction conditions similar to those described hereinbefore in respect of preparation of compounds of formula I (process (xxiii)) above;

(v) for compounds of formula II in which X¹ represents —N(R⁸)-J-R⁹, reaction of a compound of formula XXIX as hereinbefore defined, with a compound of formula VI in which X^{1b} represents —N(R⁸)-J-R⁹ and R⁸, R⁹ and J are as hereinbefore defined, for example under conditions similar to those described hereinbefore in respect of preparation of compounds of formula I (process (xxiv)) above;
(w) for compounds of formula II in which X¹ represents -Q-X², Q represents a single bond and X² represents C₁₋₈ alkyl or heterocycloalkyl substituted α to the indole ring by a G¹ substituent in which G¹ represents -A¹-R^{12a}, A¹ represents —OA⁵-, A⁵ represents a single bond and R^{12a} represents H, reaction of a corresponding compound of formula II in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O, for example under reaction conditions similar to those described hereinbefore in respect of preparation of compounds of formula I (process (xxv)) above;
(x) for compounds of formula II in which X¹ represents -Q-X², Q represents a single bond and X² represents C₂₋₈ alkyl substituted (e.g. a to the indole ring) by a G¹ substituent in which G¹ represents -A¹-R^{12a}, A¹ represents —OA⁵-, A⁵ represents a single bond and R^{12a} represents H, reaction of a corresponding compound of formula II in which X² represents C₁₋₇ alkyl substituted (e.g. α to the indole ring) by a Z¹ group in which Z¹ represents ═O, with the corresponding Grignard reagent derivative of a compound of formula V in which L² represents chloro, bromo or iodo, Q^{a} is a single bond and X² represents C₁₋₇ allyl, under conditions known to those skilled in the art;
(y) for compounds of formula II in which X¹ represents -Q-X², Q represents a single bond, and X² represents C₁₋₈ alkyl or heterocycloalkyl, both of which are unsubstituted in the position a to the indole ring, reduction of a corresponding compound of formula II in which X² represents C₁₋₈ allyl substituted a to the indole ring by a G¹ substituent in which G¹ represents -A¹-R^{12a}, A¹ represents —OA⁵-, A⁵ represents a single bond and R^{12a} represents H, for example under reaction conditions similar to those described hereinbefore in respect of preparation of compounds of formula I (process (xxvii)) above; or
(z) for compounds of formula II in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula II in which X² represents C₁₋₈ alkyl or heterocycloalkyl, which groups are substituted by one or more Z¹ groups in which Z¹ represents ═O, for example under reaction conditions similar to those described hereinbefore in respect of preparation of compounds of formula I (process (xxviii)) above.

Compounds of formula IV may be prepared as follows:
(a) reaction of a compound of formula XXIII as hereinbefore defined with a compound of formula XXX,

R¹L² XXX
wherein R¹ and L² are as hereinbefore defined or a compound of formula III as hereinbefore defined, for example under reaction conditions similar to those described hereinbefore in respect of preparation of compounds of formula I (processes (ii) and (i), respectively) above; or
(b) for compounds of formula IV wherein L¹ represents a sulfonate group, reaction of a compound of formula XXI as hereinbefore defined with an appropriate reagent for the conversion of the hydroxyl group to the sulfonate group (e.g. tosyl chloride, mesyl chloride, triflic anhydride and the like) under conditions known to those skilled in the art.

Compounds of formula VII may be prepared as follows:
(a) for compounds of formula VII in which D represents a single bond —C(O)—, —C(R¹⁰)(R¹¹)—, C₂₋₄ alkylene or —S(O)₂—, reaction of a compound of formula X,

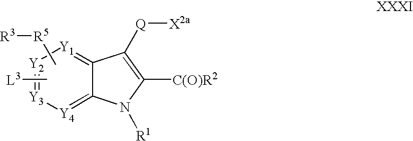

wherein Q, X^{2a}, L³, R¹, R², Y₁ to Y₄ and R³-R⁵ are as hereinbefore defined (L³ in particular may represent halo, such as bromo) with a compound of formula XI as hereinbefore defined (in which L⁴ may in particular represent —B(OH₂)), for example under reaction conditions similar to those described hereinbefore in respect of preparation of compounds of formula I (process (x)) above;
(b) reaction of a compound of formula XXIV as hereinbefore defined with a compound of formula III as hereinbefore defined, for example under reaction conditions similar to those described hereinbefore in respect of preparation of compounds of formula I (process (i)) above); or
(c) for compounds of formula VII in which Q represents a single bond and X^{2a} represents —CHO, reaction of a corresponding compound of formula I in which X¹ represents H with a mixture of DMF and, for example, oxalyl chloride, phosgene or P(O)Cl₃ (or the like) in an appropriate solvent system (e.g. DMF or dichloromethane).

Compounds of formula X may be prepared by reaction of a compound of formula XXV as hereinbefore defined, with a compound of formula III as hereinbefore defined, for example under reaction conditions similar to those described hereinbefore in respect of preparation of compounds of formula I (process (i)) above.

Compounds of formula X in which L³ represents L² may be prepared by reaction of a compound of formula X in which L³ represents L¹, with an appropriate reagent for the conversion of the L¹ group to the L² group. This conversion may be performed by methods known to those skilled in the art, for example, compounds of formula X, in which L³ is 4,4,5,5-tetramethyl-1,3,2-dioxaborolan-2-yl may be prepared by reaction of the reagent bis(pinacolato)diboron with a compound of formula X in which L³ represents L¹, for example under reaction conditions similar to those described hereinbefore in respect of preparation of compounds of formula I (process (ii)) above).

Compounds of formulae XV and XXVII may be prepared by reaction of a corresponding compound of formula IV, or XXIII, respectively, with a compound of formula XXXII,

R⁸NH₂ XXII

wherein R⁸ is as hereinbefore defined, for example under reaction conditions similar to those described hereinbefore in respect of preparation of compounds of formula I (process (ii) above).

Compounds of formulae XVII and XXVIII in which L⁵ represents an appropriate alkali metal, such as lithium may be prepared by reaction of a compound of formula XXXIII,

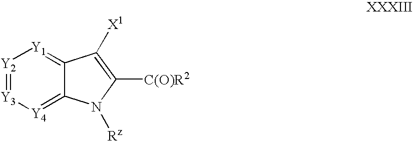

wherein R^{z} represents R¹ (in the case of a compound of formula XVII) or PG (in the case of a compound of formula XXVIII), and PG, X¹, R³ and Y₁ to Y₄ are as hereinbefore defined, with an appropriate base, such lithium diisopropylamide or BuLi under standard conditions. Compounds of formulae XVII and XXVIII in which L⁵ represents —Mg-halide may be prepared from a corresponding compound of formula XVII or XXVIII (as appropriate) in which L⁵ represents halo, for example under conditions such as those described hereinbefore in respect of process step (x). Compounds of formulae XVII and XXVIII in which L⁵ represents, for example, a zinc-based group, halo or a boronic acid group may be prepared by reacting a corresponding compound of formula XVII or XXVIII in which L⁵ represents an alkali metal with an appropriate reagent for introduction of the relevant group, for example by a metal exchange reaction (e.g. a Zn transmetallation), by reaction with a suitable reagent for the introduction of a halo group (for example, a reagent described hereinbefore in respect of preparation of compounds of formula I (process (xvi)) or, for the introduction of a boronic acid group, reaction with, for example, boronic acid or a protected derivative thereof (e.g. bis(pinacolato)diboron or triethyl borate) followed by (if necessary) deprotection under standard conditions.

Compounds of formula XXIII may be prepared by standard techniques. For example compounds of formula XXIII in which D represents a single bond —C(O)—, —C(R¹⁰)(R¹¹)—, C₂₋₄ allylene or —S(O)₂—, may be prepared by reaction of a compound of formula XXXIV,

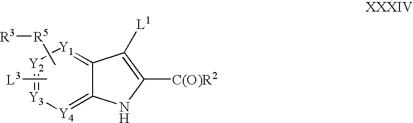

wherein L¹, L³, R²,Y₁ to Y₄ and R³-R⁵ are as hereinbefore defined with a compound of formula XI as hereinbefore defined, for example under reaction conditions similar to those described hereinbefore in respect of preparation of compounds of formula I (process (x)) above.

Compounds of formulae XXIV and XXXI, in which Q represents a single bond and X^{2a} represents —CHO, may be prepared from compounds of formulae II, or X, respectively, in which X¹ represent H, by reaction with a mixture of DMF and, for example, oxalyl chloride, phosgene or P(O)Cl₃ (or the like) in an appropriate solvent system (e.g. DMF or dichloromethane) for example as described hereinbefore.

Compounds of formulae III, V, VI, VIII, IXA, IXB, IXC, XI, XII, XIII, XIV, XVI, XVIII, XIX, XX, XXI, XXII, XXV, XXVI, XXIX, XXX, XXXII, XXXIII and XXXIV are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

Pyrrolopyridines of formulae II, IV, VII, X, XIII, XV, XVII, XXI, XXIII, XXIV, XXV, XXVI, XXVII, XXVIII, XXIX, XXXI, XXXIII and XXXIV may also be prepared with reference to a standard heterocyclic chemistry textbook (e.g. “Heterocyclic Chemistry” by J. A. Joule, K. Mills and G. F. Smith, 3^{rd} edition, published by Chapman & Hall or “Comprehensive Heterocyclic Chemistry II” by A. R. Katritzky, C. W. Rees and E. F. V. Scriven, Pergamon Press, 1996) and/or made according to the following general procedures.

For example, compounds of formulae II, XXV and XXVI in which X¹ represents H, R² represents —OR^{6a} and R^{6a} represents optionally substituted C₁₋₈ (e.g. C₁₋₆) alkyl may prepared by:
(a) cyclisation of a compound of formula XXXV,

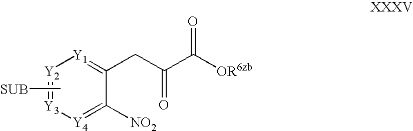

wherein SUB represents the substitution pattern that is present in the relevant compound to be formed, R^{6zb} represents optionally substituted C₁₋₈ (e.g. C₁₋₆) alkyl and Y₁ to Y₄ are as hereinbefore defined, for example under standard reductive conditions (e.g. H₂/Pd—C);
(b) reaction of a compound of formula XXXVI,

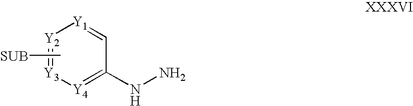

wherein SUB and Y₁ to Y₄ are as hereinbefore defined with a compound of formula XXXVII,

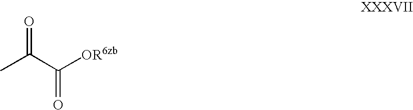

wherein R^{6zb} is as hereinbefore defined in the presence of acid; or
(c) for example when Y₂ represents —N═, reaction of a compound of formula XXXVIIA,

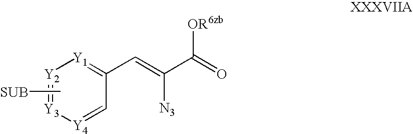

wherein SUB, Y₁ to Y₄ and R^{6zb} are as hereinbefore defined, for example under conditions such as those described in P. J. Roy et al, Synthesis, 16 (2005), 2751-2757, e.g. at elevated temperature (e.g. at reflux) in the presence of a suitable solvent (e.g. an aromatic solvent such as mesitylene or xylene).

Compounds of formula XXXV may be prepared by reaction of a compound of formula XXXVIII,

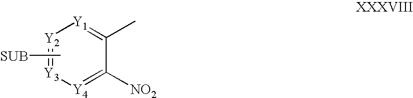

wherein SUB and Y₁ to Y₄ are as hereinbefore defined with a compound of formula XXXIX,

(C(O)OR^{6zb})₂ XXXIX

wherein R^{6zb} is as hereinbefore defined, for example in the presence of an appropriate weak base (e.g. KOR^{6zb}, such as KOEt).

Compounds of formula XXXVIIA may be prepared by reaction of a compound of formula XL,

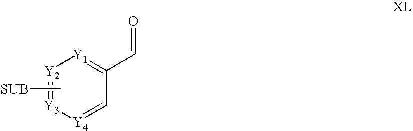

wherein SUB and Y₁ to Y₄ are as hereinbefore defined, with a compound of formula XLI,

N₃—CH₂—C(O)OR^{6zb} XLI

wherein R^{6zb} is as hereinbefore defined, for example in the presence of a suitable base (such as an alkali metal ethoxide (e.g. NaOEt)) in the presence of an alcoholic solvent (e.g. ethanol) at below room temperature (e.g. 0° C.).

Compounds of formulae XXXVI, XXXVII, XXXVIII, XXXIX, XL and XLI are either commercially available, are known in the literature, or may be obtained either by analogy with the processes described herein, or by conventional synthetic procedures, in accordance with standard techniques, from available starting materials using appropriate reagents and reaction conditions. For example, compounds of formula XXXVIII in which SUB represents the substitution pattern of a compound of formula XXV, and one of e.g. R³, R⁴ or R⁵ represents -D-E and D represents —O— or —S—, may be prepared by reaction of a corresponding compound of formula XXXVIII in which L³ represents, for example, halo, with a compound of formula XII as hereinbefore defined or a phenol equivalent thereof (i.e. a compound of formula E-OH), under known reaction conditions. In this respect, the skilled person may refer to inter alia “Comprehensive Organic Synthesis” by B. M. Trost and I. Fleming, Pergamon Press, 1991.

The substituents X¹, R¹, R², R³, R⁴ and R⁵ in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, and etherifications. The precursor groups can be changed to a different such group, or to the groups defined in formula I, at any time during the reaction sequence. For example, in cases where R² represents —OR^{6a} and R^{6a} does not initially represent hydrogen (so providing an ester functional group), the skilled person will appreciate that at any stage during the synthesis (e.g. the final step), the relevant substituent may be hydrolysed to form a carboxylic acid functional group (in which case R^{6a} will be hydrogen). In this respect, the skilled person may also refer to “Comprehensive Organic Functional Group Transformations” by A. R. Katritzky, O. Meth-Cohn and C. W. Rees, Pergamon Press, 1995.

Compounds of the invention may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention (including, but not limited to, compounds of formula I in which R² represents —OR^{6a} and R^{6a} is other than hydrogen) may possess no or minimal pharmacological activity as such, but may be administered parenterally or orally, and thereafter be metabolised in the body to form compounds of the invention that possess pharmacological activity as such (including, but not limited to, corresponding compounds of formula I, in which R² represents —OR^{6a} and R^{6a} represents hydrogen). Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention are particularly useful because they may inhibit the activity of a member of the MAPEG family.

Compounds of the invention are particularly useful because they may inhibit (for example selectively) the activity of prostaglandin E synthases (and particularly microsomal prostaglandin E synthase-1 (mPGES-1)), i.e. they prevent the action of mPGES-1 or a complex of which the mPGES-1 enzyme forms a part, and/or may elicit a mPGES-1 modulating effect, for example as may be demonstrated in the test described below. Compounds of the invention may thus be useful in the treatment of those conditions in which inhibition of a PGES, and particularly mPGES-1, is required.

Compounds of the invention may inhibit the activity of leukotriene C₄ (LTC₄), for example as may be shown in a test such as that described in Eur. J. Biochem., 208, 725-734 (1992), and may thus be useful in the treatment of those conditions in which inhibition of LTC₄ is required. Compounds of the invention may also inhibit the activity of 5-lipoxygenase-activating protein (FLAP), for example as may be shown in a test such as that described in Mol. Pharmacol., 41, 873-879 (1992).

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds the invention may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a member of the MAPEG family such as a PGES (such as mPGES-1), LTC₄ and/or FLAP and/or a method of treatment of a disease in which inhibition of the activity of a member of the MAPEG family such as a PGES (and particularly mPGES-1), LTC₄ and/or FLAP is desired and/or required (e.g. inflammation), which method comprises administration of a therapeutically effective amount of a compound of the invention, as hereinbefore defined, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined, in admixture with a pharmaceutically acceptable adjuvant, diluent or carrier.

Compounds of the invention may also be combined with other therapeutic agents that are useful in the treatment of inflammation (e.g. NSAIDs and coxibs).

According to a further aspect of the invention, there is provided a combination product comprising:

(A) a compound of the invention, as hereinbefore defined; and

(B) another therapeutic agent that is useful in the treatment of inflammation, wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, another therapeutic agent that is useful in the treatment of inflammation, and a pharmaceutically-acceptable adjuvant, diluent or carrier; and
(2) a kit of parts comprising components:
(a) a pharmaceutical formulation including a compound of the invention, as hereinbefore defined, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective, and preferably selective, inhibitors of a member of MAPEG family, e.g. inhibitors of prostaglandin E synthases (PGES) and particularly microsomal prostaglandin E synthase-1 (mPGES-1). The compounds of the invention may reduce the formation of the specific arachidonic acid metabolite PGE₂ without reducing the formation of other COX generated arachidonic acid metabolites, and thus may not give rise to the associated side-effects mentioned hereinbefore.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

In the assay in PGES-1 catalyses the reaction where the substrate PGH₂ is converted to PGE₂. mPGES-1 is expressed in E. coli and the membrane fraction is dissolved in 20 mM NaPi-buffer pH 8.0 and stored at −80° C. In the assay mPGES-1 is dissolved in 0.1M KPi-buffer pH 7.35 with 2.5 mM glutathione. The stop solution consists of H₂O/MeCN (7/3), containing FeCl₂ (25 mM) and HCl (0.15 M). The assay is performed at room temperature in 96-well plates. Analysis of the amount of PGE₂ is performed with reversed phase HPLC (Waters 2795 equipped with a 3.9×150 mm C18 column). The mobile phase consists of H₂O/MeCN (7/3), containing TFA (0.056%), and absorbance is measured at 195 nm with a Waters 2487 LTV-detector.

The following is added chronologically to each well:
1. 100 μL mPGES-1 in KPi-buffer with glutathione. Total protein concentration: 0.02 mg/mL.
2. 1 μL inhibitor in DMSO. Incubation of the plate at room temperature for 25 minutes.
3. 4 μL of a 0.25 mM PGH₂ solution. Incubation of the plate at room temperature for 60 seconds.
4. 100 μL stop solution.
   180 μL per sample is analyzed with HPLC.

### EXAMPLES

The invention is illustrated by way of the following examples. Starting materials and chemical reagents specified in the syntheses described below are commercially available from, e.g. Sigma-Aldrich Fine Chemicals.

### Example 1

### 1-(4-Isopropoxyphenyl)-5-(3-trifluoromethylphenoxy)pyrrolo[2,3-c]pyridine-2-carboxylic Acid

### (a) 4-Methyl-5-nitro-2-(3-trifluoromethylphenoxy)pyridine

Finely ground NaOH (106 mg, 2.66 mmol) was added to a solution of 2-chloro-4-methyl-5-nitropyridine (345 mg, 2.0 mmol) and 3-trifluoromethylplienol (270 μL, 2.2 mmol) in anhydrous MeCN (6 mL). The nature was heated at reflux for 4 h and cooled to rt. H₂O (6 mL) was added and the mixture was extracted with EtOAc (3×15 mL). The combined extracts were washed with H₂O and brine, dried (Na₂SO₄), concentrated and purified by chromatography to afford the sub-title compound (550 mg, 93%).

### (b) 3-[5-Nitro-2-(3-trifluoromethoxyphenoxy)pyridin-4-yl]-2-oxopropionic acid ethyl ester

Potassium (126 mg, 3.2 mmol) was added under argon to a mixture of anhydrous Et₂O (9 mL) and absolute EtOH (720 μL). After all potassium was dissolved, a solution of oxalic acid diethyl ester (450 μL, 3.3 mmol) in anhydrous Et₂O (2 mL) was added via syringe. After 10 min, 4-methyl-5-nitro-2-(3-trifluoromethyl-phenoxy)pyridine (920 mg, 3.0 mmol; see step (a) above) in anhydrous Et₂O (5 mL) was added and the mixture was stirred at rt for 48 h. The dark red precipitate was collected, washed with Et₂O and dried. The solid was suspended in water (15 mL) and the pH was adjusted to 3-4 with AcOH. The mixture was extracted with CH₂Cl₂ (2×30 mL) and the combined extracts were washed with H₂O and brine, dried (Na₂SO₄), concentrated and purified by chromatography to afford the sub-title compound (560 mg, 47%).

### (c) 5-(3-Trifluoromethylphenoxy)pyrrolo[2,3-c]pyridine-2-carboxylic acid ethyl ester

3-[5-Nitro-2-(3-trifluoromethoxyphenoxy)pyridin-4-yl]-2-oxopropionic acid ethyl ester (1.40 g, 3.5 mmol; see step (b) above) in EtOAc (14 mL) and EtOH (14 mL) was hydrogenated at ambient temperature and pressure over Pd—C (10%, 320 mg, 0.3 mmol) for 2 h. Filtration, concentration and purification by chromatography afforded the sub-title compound (1.01 g, 82%).

### (d) 1-(4-Isopropoxyphenyl)-5-(3-trifluoromethylphenoxy)pyrrolo[2,3-c]pyridine-2-carboxylic acid ethyl ester

Anhydrous CH₂Cl₂ (80 mL), followed by Et₃N (280 μL, 2.0 mmol) and pyridine (160 μL, 2.0 mmol) were added to 5-(3-trifluoromethylphenoxy)pyrrolo[2,3-c]pyridine-2-carboxylic acid ethyl ester (350 mg, 1.0 mmol; see step (c) above), Cu(OAc)₂ (360 mg, 2.0 mmol), 4 Å molecular sieves (ca. 20 mg) and 4-isopropoxyphenylboronic acid (360 mg, 2.0 mmol). The mixture was stirred vigorously at rt for 72 h and filtered through Celite®. The solids were washed with EtOAc, and the filtrates concentrated and purified by chromatography to afford the sub-title compound (310 mg, 65%).

### (e) 1-(4-Isopropoxyphenyl)-5-(3-trifluoromethylphenoxy)pyrrolo[2,3-c]pyridine-2-carboxylic acid

A mixture of 1-(4-isopropoxyphenyl)-5-(3-trifluoromethylphenoxy)pyrrolo[2,3-c]-pyridine-2-carboxylic acid ethyl ester (310 mg, 0.6 mmol, see step (d) above), NaOH (aq, 2 M, 2 mL) and dioxane (3 mL) was heated at 120° C. for 30 min. The mixture was diluted with H₂O and acidified with HCl (aq, 1 M) to pH 5. The precipitate was collected and recrystallised from EtOH to give the title compound (170 mg, 63%).

200 MHz ¹H-NMR (DMSO-d₆, ppm) δ 13.34-13.29 (1H, br s) 8.12-8.09 (1H, m) 7.63-7.51 (1H, m) 7.49-7.41 (2H, m) 7.39-7.25 (5H, m) 7.06-6.98 (2H, m) 4.68 (1H, septet, J=5.9 Hz) 1.30 (6H, d, J=5.9 Hz).

### Example 2

### 1-(4-Isopropoxyphenyl)-4,6-bis-(4-trifluoromethylphenyl)pyrrolo [3,2-c]pyridine-2-carboxylic acid

### (a) 2,6-Dichloropyridine-3-carboxaldehyde

BuLi (2.5 M in hexanes, 36 mL, 90 mmol) was slowly added to diisopropylamine (12.5 mL, 89.2 mmol) in THF (80 mL) at 0° C. The mixture was cooled to −80° C. and 2,6-dichloropyridine (12.0 g, 81.1 mmol) in THF (80 mL) was added over 70 min at −80° C. After 30 min, DMF (19 mL, 243 mmol) was added and the mixture was stirred for an additional 40 min at −80° C. NH₄Cl (aq, sat) was added and the mixture was extracted with EtOAc. The combined extracts were dried (Na₂SO₄), concentrated and crystallised from hexane to give the sub-title compound (7.43 g 52%).

### (b) (Z)-2-Azido-3-(2,6-dichloropyridin-3-yl)acrylic acid ethyl ester

NaOEt (2.5 M in EtOH, 28.8 mL, 72 mmol) was added to 2,6-dichloropyridine-3-carboxaldehyde (3.17 g, 18.0 mmol; see step (a) above) and azidoacetic acid ethyl ester (9.3 g, 72 mmol) in EtOH (130 mL) at 0° C. during 40 min. After 3 h at 0° C., NH₄Cl (aq, sat) was added and the mixture was extracted with EtOAc. The combined extracts were dried (Na₂SO₄), concentrated and purified by chromatography to afford the sub-title compound (1.7 g, 33%).

### (c) 4,6-Dichloropyrrolo[3,2-c]pyridine-2-carboxylic acid ethyl ester

(Z)-2-Azido-3-(2,6-dichloropyridin-3-yl)acrylic acid ethyl ester (1.5 g, 5.23 mmol; see step (b) above) in mesitylene (15 mL) was added to mesitylene (50 mL) at reflux. After 1.5 h at reflux, the mixture was concentrated and the residue purified by chromatography to afford the sub-title compound (310 mg, 23%).

### (d) 4,6-Dichloro-1-(4-isopropoxyphenyl)pyrrolo[3,2-c]pyridine-2-carboxylic acid ethyl ester

The sub-title compound was prepared in accordance with Example 1, step (d), from 4,6-dichloropyrrolo[3,2-c]pyridine-2-carboxylic acid ethyl ester (293 mg, 1.13 mmol; see step (c) above) and 4-isopropoxyphenylboronic acid (407 mg, 2.26 mmol), reaction time 8 h. Yield 373 g (84%).

### (e) 1-(4-Isopropoxyphenyl)-4,6-bis-(4-trifluoromethylphenyl)pyrrolo[3,2-c]pyridine-2-carboxylic acid ethyl ester

A mixture of 4,6-dichloro-1-(4-isopropoxyphenyl)pyrrolo[3,2-c]pyridine-2-carboxylic acid ethyl ester (320 mg, 0.814 mmol) (step (d) above), 4-trifluoromethylphenylboronic acid (618 mg, 3.25 mmol), K₃PO₄ (1.20 g, 5.6 mmol), Pd(PPh₃)₄ (47 mg, 0.04 mmol) and toluene (3 mL) was stirred at 85° C. for 48 h. The mixture was cooled to rt, diluted with EtOAc, washed with H₂O and brine, dried (Na₂SO₄), concentrated and purified by chromatography to afford the sub-title compound (281 mg, 56%).

### (f) 1-(4-Isopropoxyphenyl)-4,6-bis(4-trifluoromethylphenyl)pyrrolo[3,2-c]pyridine-2-carboxylic acid

A mixture of 1-(4-isopropoxyphenyl)-4,6-bis(4-trifluoromethylphenyl)pyrrolo-[3,2-c]pyridine-2-carboxylic acid ethyl ester (110 mg, 0.18 mmol; see step (e) above), NaOH (72 mg, 1.8 mmol), H₂O (6 mL) and EtOH (4 mL) was stirred at reflux for 1.5 h. After cooling, the EtOH was partly evaporated, and the mixture was acidified with HCl (aq, 1 M) to pH 5 and extracted with EtOAc. The combined extracts were washed with brine, dried (Na₂SO₄), concentrated and purified by chromatography to give the title compound (100 mg, 95%).

200 MHz ¹H-NMR (DMSO-d₆, ppm) 13.4-13.0 (1H br s) 8.44-8.29 (4H, m) 8.06-7.94 (2H, m) 7.88-7.77 (2H, m) 7.68 (1H, s) 7.58 (1H, s) 7.46-7.36 (2H, m) 7.15-7.04 (2H, m) 4.74 (1H, septet, J=6.0 Hz) 1.36 (6H, d, J=6.0 Hz).

### Example 3

### 6-Chloro-1-(4-isopropoxyphenyl)-4-(4-trifluoromethylphenyl)pyrrolo[3,2-c]pyridine-2-carboxylic acid

### (a) 6-Chloro-1-(4-isopropoxyphenyl)-4-(4-trifluoromethylphenyl)pyrrolo[3,2-c]-pyridine-2-carboxylic acid ethyl ester

4,6-Dichloro-1-(4-isopropoxyphenyl)pyrrolo[3,2-c]pyridine-2-carboxylic acid ethyl ester (110 mg, 0.28 mmol) (step (d), Example 2), 4-trifluoromethyl-phenylboronic acid (319 mg, 1.68 mmol), Na₂CO₃ (aq, 2 M, 1.0 mL, 2.0 mmol), Pd(PPh₃)₄ (16 mg, 0.015 mmol), toluene (1 mL) and EtOH (0.23 mL) was stirred at 85° C. for 6 h. The mixture was cooled to rt, diluted with EtOAc, washed with H₂O and brine, dried (Na₂SO₄), concentrated and purified by chromatography to afford the sub-title compound (70 mg, 49%).

### (b) 6-Chloro-1-(4-isopropoxyphenyl)-4-(4-trifluoromethylphenyl)pyrrolo[3,2-c]-pyridine-2-carboxylic acid

The title compound was prepared in accordance with step ((e) in Example 1) from 6-chloro-1-(4-isopropoxyphenyl)-4-(4-trifluoromethylphenyl)pyrrolo[3,2-c]pyridine-2-carboxylic acid ethyl ester (see step (a) in above).

200 MHz ¹H-NMR (DMSO-d₆, ppm) δ 8.28-8.17 (2H, m) 7.99-7.88 (2H m) 7.05 (1H, s) 7.36-7.26 (2H, m) 7.09-6.96 (2H, m) 6.99 (1H, s) 4.68 (1H, m) 1.32 (6H, d, J=6.0 Hz).

### Example 4

### 1-(4-Isopropoxyphenyl)-4,6-bis(3-trifluoromethylphenoxy)pyrrolo[3,2-c]pyridine-2-carboxylic acid

### (a) 2,6-Bis(3-trifluoromethylphenoxy)pyridine-3-carboxaldehyde

A mixture of 2,6-dichloropyridine-3-carboxaldehyde (1.0 g, 5.68 mmol; see step (a) Example 2), 3-trifluoromethylphenol (3.68 g, 22.7 mmol), K₂CO₃ (3.99 g, 28.4 mmol), 18-crown-6 (150 mg, 0.57 mmol) and DMF (6 mL) was stirred at 60° C. for 3 h. The mixture was cooled to rt, diluted with H₂O and extracted with t-BuOMe. The combined extracts were washed with H₂O and brine, dried (Na₂SO₄) and concentrated. The residue was dissolved in 10 mL EtOH and this solution was slowly added to NaOH (aq, 10%) at 0° C. After 10 min, the precipitate was filtered off, washed with H₂O and dried to afford the sub-title compound (1.15 g, 47%).

### (b) (Z)-2-Azido-3-[2,6-bis(3-trifluoromethylphenoxy)pyridin-3-yl]acrylic acid ethyl ester

NaOEt (2.5 M in EtOH, 1.5 mL, 3.74 mmol) was added to 2,6-bis(3-trifluoromethylphenoxy)pyridine-3-carboxaldehyde (400 mg, 3.74 mmol; see step (a) above) and azidoacetic acid ethyl ester (483 mg, 3.74 mmol) in EtOH (1.7 mL) at 0° C. during 40 min. After 3 h at 0° C., NH₄Cl (aq, sat) was added and the solid formed was filtered off, washed with H₂O and dissolved in CH₂Cl₂. The solution was dried (MgSO₄) and concentrated to give the sub-title compound (368 mg, 72%).

### (c) 4,6-Bis(3-trifluoromethylphenoxy)pyridine[3,2-c]pyridine-2-carboxylic acid ethyl ester

(Z)-2-Azido-3-[2,6-bis(3-trifluoromethylphenoxy)pyridin-3-yl]acrylic acid ethyl ester (290 mg, 0.54 mmol; see step (b) above) in xylene (1 mL) was added to xylene (4 μl) at reflux. After 1 h at reflux, the mixture was cooled to 0° C. and the precipitate collected and washed with cold xylene to afford the sub-title compound (156 mg, 57%).

### (d) 1-(4-isopropoxyphenyl)-4,6-bis(3-trifluoromethylphenoxy)pyrrolo[3 2-cl-pyridine-2-carboxylic acid ethyl ester

The sub-title compound was prepared in accordance with step (d) in Example 1 from 4,6-bis(3-trifluoromethylphenoxy)pyrrolo[3),2-c]pyridine-2-carboxylic acid ethyl ester (see step (c) in above).

### (e) 1-(4-Isopropoxyphenyl)-4,6-bis(3-trifluoromethylphenoxy)pyrrolo[3,2-c]-pyridine-2-carboxylic acid

The title compound was prepared in accordance with step (e) in Example 1 from 1-(4-isopropoxyphenyl)-4,6-bis(3-trifluoromethylphenoxy)pyrrolo[3,2-c]pyridine-2-carboxylic acid ethyl ester (see step (d) in above).

200 MHz ¹H-NMR (DMS O-d₆, ppm) δ 7.60-7.49 (4H, m) 7.48-7.36 (3H, m) 7.31-7.24 (4H, m) 7.09-6.98 (2H, m) 6.33 (1H, s) 4.69 (1H, septet, J=6.0 Hz) 1.32 (6H, d, J=6.0 Hz).

### Example 5

The following compounds are prepared in accordance with techniques described herein:

### 5-(3-chlorophenoxy)-1-(4-isopropoxyphenyl)pyrrolo[3,2-b]pyridine-2-carboxylic acid;

1-(4-isopropoxyphenyl)-5-(4-trifluoromethylphenyl)pyqrolo [3,2-b]-pyridine-2-carboxylic acid;
3-chloro-5-(3-chlorophenoxy)-1-(4-isopropoxyphenyl)pyrrolo[3,2-b]-pyridine-2-carboxylic acid;
3-chloro-1-(4-isopropoxyphenyl)-5-(4-trifluoromethylphenyl)pyirolo-[3,2-b]pyridine-2-carboxylic acid;
5-(3-chlorophenoxy)-3-(2-cyanoethyl)-1-(4-isopropoxyphenyl)pyrrolo-[3,2-b]pyridine-2-carbox)ylic acid;
5-(3-chlorophenoxy)-1-(4-isopropoxyphelayl)-3-[2-(pyridin-2-yl)ethyl]pyyrolo-[3,2-b]pyridine-2-carboxylic acid;
5-(3-chlorophelnoxy)-1-(4-isopropoxyphenyl)-3-(pentanamido)pyrrolo-[3,2-b]pyridine-2-carboxylic acid;
3-(4-chlorobenzamido)-5-(3-chlorophenoxy)-1-(4-isopropoxyphenyl)pyrrolo[3,2-b]pyridine-2-carboxylic acid;
5-(3-chlorophenoxy)-1-(4-isopropoxyphenyl)-3-(2-oxopyrrolidin-1-yl)pyrrolo-[3,2-b]pyridine-2-carboxylic acid;
1-(4-cyclopropoxyphenyl)-4,6-bis(5-trifluoromethylpyridin-2-yl)pyrrolo[3,2-c]pyridine-2-carboxylic acid;
4,6-bis(4-cyclohexylphenyl)-1-(4-isopropoxyphenyl)pyrrolo[3,2-c]pyridine-2-carboxylic acid;
1-(4-isopropoxyphenyl)-6-(5-trifluoromethylpyridin-2-yl)pyrrolo[3,2-c]pyridine-2-carboxylic acid;
1-(4-isopropoxyphenyl)-4-methoxy-6-(5-trifluoromethylpyridin-2-yl)pyrrolo-[3,2-c]pyridine-2-carboxylic acid;
6-(4-cyclohexylphenyl)-1-(4-cyclopropoxyphenyl)-4-(dimethylamino)-pyrrolo-[3,2-c]pyridine-2-carboxylic acid;
1-(4-cyclopropoxyphenyl)-6-(2-trifluoromethylpyridin-4-yloxy)pyrrolo-[3,2-c]pyridine-2-carboxylic acid;
1-(4-isopropoxyphenyl)-5-(4-trifluoromethylphenyl)pyrrolo[2,3-c]pyridine-2-carboxylic acid;
5-(3-chlorophenoxy)-1-(4-isopropoxyphenyl)pyrrolo[2,3-c]pyridine-2-carboxylic acid;
3-chloro-1-(4-isopropoxyphenyl)-5-(4-trifluoromethylphenyl)pyrrolo-[2,3-c]pyridine-2-carboxylic acid;
3-chloro-5-(3-chlorophenoxy)-1-(4-isopropoxyphenyl)pyrrolo[2,3-c]pyridine-2-carboxylic acid;
1-(4-isopropoxyphenyl)-5-(5-trifluoromethylpyridin-2-yl)pyrrolo[2,3-c]pyridine-2-carboxylic acid;
1-(4-isopropoxyphenyl)-5-(4-trifluoromethylphenyl)pyrrolo[2,3-b]-pyridine-2-carboxylic acid;
5-(4-cyclohexylphenyl)-1-(4-cyclopropoxyphenyl)pyrrolo[2,3-b]-pyridine-2-carboxylic acid;
1-(4-isopropoxyphenyl)-5-(5-trifluoromethylpyridin-2-yl)pyrrolo[2,3-b]pyridine-2-carboxylic acid;
3-chloro-1-(4-isopropoxyphenyl)-5-(4-trifluoromethylphenyl)pyrrolo-[2,3-b]pyridine-2-carboxylic acid;
3-chloro-5-(4-cyclohexylphenyl)-1-(4-cyclopropoxyphenyl)pyrrolo-[2,3-b]pyridine-2-carboxylic acid;
3-chloro-1-(4-isopropoxyphenyl)-5-(5-trifluoromethylpyridin-2-yl)pyrrolo[2,3-b]pyridine-2-carboxylic acid;
3-isopropoxy-1-(4-isopropoxyphenyl)-5-(4-trifluoromethylphenyl)pyrrolo[2,3-b]pyridine-2-carboxylic acid;
3-(carboxymethoxy)-5-(4-cyclohexylphenyl)-1-(4-cyclopropoxyphenyl)pyrrolo-[2,3-b]pyridine-2-carboxylic acid; and
3-(2-aminoethoxy)-1-(4-isopropoxyphenyl)-5-(5-trifluoromethylpyridin-2-yl)-pyrrolo[2,3-b]pyridine-2-carboxylic acid.

### Example 6

Title compounds of the examples were tested in the biological test described above and were found to exhibit 50% inhibition of in mPGES-1 at a concentration of 10 μM or below. For example, the following representative compounds of the examples exhibited the following IC₅₀ values:

| | |
|---|---|
| Example 1: | 2300 nM |
| Example 3: | 750 nM |

1. A compound of formula I, 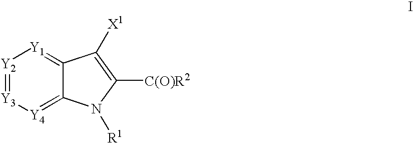
wherein
one of Y₁, Y₂, Y₃ and Y₄ represents —N═ and the others respectively represent —C(R³)═, —C(R⁴)═ and —C(R⁵)═;
R² represents —OR^{6a}, or —N(R^{6b})R⁷;
X¹ represents H, halo, —N(R⁸)-J-R⁹ or -Q-X²;
J represents a single bond, —C(O)— or —S(O)ₘ—;
Q represents a single bond, —O—, —C(O)— or —S(O)ₘ—;
X² represents:
(a) C₁₋₈ alkyl or a heterocycloalkyl group, both of which are optionally substituted by one or more substituents selected from G¹ and/or Z¹; or
(b) an aryl group or a heteroaryl group, both of which are optionally substituted by one or more substituents selected from A;
one of the groups R³, R⁴ and R⁵ represents -D-E and:
a) the other groups are independently selected from hydrogen, G¹, an aryl group, a heteroaryl group (which latter two groups are optionally substituted by one or more substituents selected from A), C₁₋₈ alkyl and a heterocycloalkyl group (which latter two groups are optionally substituted by one or more substituents selected from G¹ and/or Z¹); and/or
b) any two other groups which are adjacent to each other are optionally linked to form, along with two carbon atoms of the essential pyridine ring in the compound of formula I, a 3- to 8-membered ring, optionally containing 1 to 3 heteroatoms, which ring is itself optionally substituted by one or more substituents selected from halo, —R^{6c}, OR^{6d} and ═O;
D represents a single bond, —O—, —C(R¹⁰)(R¹¹)—, C₂₋₄ alkylene, —C(O)— or —S(O)ₘ—;
m represents 0, 1 or 2;
R¹ and E independently represent an aryl group or a heteroaryl group, both of which groups are optionally substituted by one or more substituents selected from A;
R^{6a}, R^{6b}, R^{6c}, R^{6d}, R⁷, R⁸ and R⁹ independently represent:
I) hydrogen;
II) an aryl group or a heteroaryl group, both of which are optionally substituted by one or more substituents selected from B; or
III) C₁₋₈ alkyl or a heterocycloalkyl group, both of which are optionally substituted by one or more substituents selected from G¹ and/or Z¹; or
R^{6b} and R⁷, and R⁸ and R⁹ may be linked together to form, along with the N atom and (in the case of R⁹) the J group to which they are attached, a 3- to 8-membered ring, optionally containing 1 to 3 heteroatoms and/or 1 to 3 double bonds, which ring is optionally substituted by one or more substituents selected from G¹ and/or Z¹;
R¹⁰ and R¹¹ independently represent H, halo or C₁₋₆ alkyl, which latter group is optionally substituted by halo, or R¹⁰ and R¹¹ may together form, along with the carbon atom to which they are attached, a 3- to 6-membered ring, which ring optionally contains a heteroatom and is optionally substituted by one or more substituents selected from halo and C₁₋₃ alkyl, which latter group is optionally substituted by one or more halo substituents;
A represents:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents halo, cyano, —N₃, —NO₂, —ONO₂ or -A¹-R^{12a};
wherein A¹ represents a single bond or a spacer group selected from —C(O)A²-, —S(O)₂A³-, —N(R^{13a})A⁴- or -A⁵-, in which:
A² represents a single bond, —O—, —N(R^{13b})— or —C(O)—;
A³ represents a single bond, —O— or —N(R^{13c})—;
A⁴ and A⁵ independently represent a single bond, —C(O)—, —C(O)N(R^{13d})—, —C(O)O—, —S(O)₂— or —S(O)₂N(R^{13e})—;
Z¹ represents ═O, ═S, ═NOR^{12b}, ═NS(O)₂N(R^{13f})R^{12c}, ═NCN or ═C(H)NO₂;
B represents:
I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;
G² represents halo, cyano, —N₃, —NO₂, —ONO₂ or -A⁶-R^{14a};
wherein A⁶ represents a single bond or a spacer group selected from
—C(O)A⁷-, —S(O)₂A⁸-, —N(R^{15a})A⁹- or —OA¹⁰-, in which:
A⁷ represents a single bond, —O—, —N(R^{15b})— or —C(O)—;
A⁸ represents a single bond, —O— or —N(R^{15c})—;
A⁹ and A¹⁰ independently represent a single bond, —C(O)—, —C(O)N(R^{15d})—,
—C(O)O—, —S(O)₂— or —S(O)₂N(R^{15e})—;
Z² represents ═O, ═S, ═NOR^{14b}, ═NS(O)₂N(R^{15f})R^{14c}, ═NCN or ═C(H)NO₂;
R^{12a}, R^{12b}, R^{12c}, R^{13a}, R^{13b}, R^{13c}, R^{13d}, R^{13e}, R^{13f}, R^{14a}, R^{14b}, R^{14c}, R^{15a}, R^{15b}, R^{15c}, R^{15d}, R^{15e} and R^{15f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{12a} to R^{12c} and R^{13a} to R^{13f}, and/or R^{14a} to R^{14c} and R^{15a} to R^{15f}, may be linked together to form a further 3- to 8-membered ring, optionally containing 1 to 3 heteroatoms and/or 1 to 3 double bonds, which ring is optionally substituted by one or more substituents selected from G³ and/or Z³;
G³ represents halo, cyano, —N₃, —NO₂, —ONO₂ or -A¹¹-R^{16a};
wherein A¹¹ represents a single bond or a spacer group selected from —C(O)A¹²-, —S(O)₂A¹³-, —N(R^{17a})A¹⁴ or —OA¹⁵-, in which:
A¹² represents a single bond, —O—, —N(R^{17b})— or —C(O)—;
A¹³ represents a single bond, —O— or —N(R^{17c})—;
A¹⁴ and A¹⁵ independently represent a single bond, —C(O)—, —C(O)N(R^{17d})—, —C(O)O—, —S(O)₂— or —S(O)₂N(R^{17e})—;
Z³ represents ═O, ═S, ═NOR^{16b}, ═NS(O)₂N(R^{17f})R^{16c}, ═NCN or ═C(H)NO₂;
R^{16a}, R^{16b}, R^{16c}, R^{17a}, R^{17b}, R^{17c}, R^{17d}, R^{17e} and R^{17f} are independently selected from:
i) hydrogen;
ii) C₁₋₆ alkyl or a heterocycloalkyl group, both of which groups are optionally substituted by one or more substituents selected from halo, C₁₋₄ alkyl, —N(R^{18a})R^{19a}, —OR^{18b} and ═0; and
iii) an aryl or heteroaryl group, both of which are optionally substituted by one or more substituents selected from halo, C₁₋₄ alkyl, —N(R^{18c})R^{19b} and —OR^{18d}; or
any pair of R^{16a} to R^{16c} and R^{17a} to R^{17f} may be linked together to form a further 3- to 8-membered ring, optionally containing 1 to 3 heteroatoms and/or 1 to 3 double bonds, which ring is optionally substituted by one or more substituents selected from halo, C₁₋₄ alkyl, —N(R^{18e})R^{19c}, —OR^{18f} and ═O;
R^{18a}, R^{18b}, R^{18c}, R^{18d}, R^{18e}, R^{18f}, R^{19a}, R^{19b} and R^{19c} are independently selected from hydrogen and C₁₋₄ alkyl, which latter group is optionally substituted by one or more halo groups;
or a pharmaceutically-acceptable salt thereof.

2. A compound as claimed in claim 1, wherein A represents G¹ or C₁₋₆ alkyl optionally substituted by one or more G¹ groups.

3. A compound as claimed in claim 1 or claim 2, wherein G¹ represents halo, cyano, —NO₂ or -A¹-R^{12a}.

4. A compound as claimed in claim 1, wherein A¹ represents a single bond, —C(O)A²-, —N(R^{13a})A⁴- or —OA⁵-.

5. A compound as claimed in claim 4, wherein A¹ represents —C(O)A²-, —N(R^{13a})A⁴- or —OA⁵-.

6. A compound as claimed in claim 1, wherein A² represents —O—.

7. A compound as claimed in claim 1, wherein A⁴ and A⁵ independently represent a single bond.

8. A compound as claimed in claim 1, wherein R^{12a} to R^{12c} independently represent a phenyl group, a tetrazolyl group, an imidazolyl group, a pyridyl group (all of which are optionally substituted by one or more G³ groups), H or a C₁₋₄ alkyl group, which latter group is optionally substituted by one or more G³ groups.

9. A compound as claimed in claim 1, wherein B represents G².

10. A compound as claimed in claim 1, wherein G² represents halo.

11. A compound as claimed in claim 1, wherein G³ represents -A¹¹-R^{16a} or halo.

12. A compound as claimed in claim 11, wherein G³ represents halo.

13. A compound as claimed in claim 1, wherein A¹¹ represents a single bond.

14. A compound as claimed in claim 1, wherein R^{16a} to R^{16c} independently represent C₁₋₂ alkyl optionally substituted by one or more fluoro atoms.

15. A compound as claimed in claim 1, wherein one of R³ and R⁴ represents -D-E and the other represents H, aryl or G¹.

16. A compound as claimed in claim 15, wherein one of R³ and R⁴ represents -D-E and the other represents H or G¹.

17. A compound as claimed in claim 1, wherein D represents a single bond or —O—.

18. A compound as claimed in claim 1, wherein R¹, X² (when X² represents an aryl or heteroaryl group) and/or E represent optionally substituted phenyl, naphthyl, pyrrolyl, furanyl, thienyl, pyrazolyl, imidazolyl, oxazolyl, isoxazolyl, thiazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, groups.

19. A compound as claimed in claim 18, wherein R¹ and E independently represent optionally substituted pyridyl, phenyl or imidazolyl.

20. A compound as claimed in claim 18 or claim 19, wherein the optional substituents are selected from halo, cyano, —NO₂, C₁₋₆ alkyl (which alkyl group may be linear or branched, cyclic, part-cyclic, unsaturated and/or optionally substituted with one or more halo group), heterocycloalkyl (which heterocycloalkyl group is optionally substituted by one or more substituents selected from C₁₋₃ alkyl and ═O), —OR²⁰ and —N(R²⁰)R²¹, wherein R²⁰ and R²¹ independently represent H or linear, branched or cyclic C₁₋₆ alkyl (which alkyl group is optionally substituted by one or more halo groups).

21. A compound as claimed in claim 1, wherein R⁵ represents H.

22. A compound as claimed in claim 1, wherein X¹ represents H, halo or -Q-X².

23. A compound as claimed in claim 1, wherein Q represents —S—, —O— or a single bond.

24. A compound as claimed in claim 1, wherein X² represents C₁₋₄ alkyl optionally substituted by one or more G¹ groups.

25. A compound as claimed in claim 1, wherein J represents —C(O)—.

26. A compound as claimed in claim 25, wherein R⁸ represents H.

27. A compound as claimed in claim 1, wherein R⁹ represents H, C₁₋₅ alkyl, optionally substituted by one or more G¹ groups, or a phenyl group, optionally substituted by one or more B groups.

28. A compound as claimed in claim 1, wherein R⁸ and R⁹ are linked together to form a propylene or a butylene chain to form, together with the nitrogen atom and the J group to which they are respectively attached, a 5- or 6-membered ring.

29. A compound as claimed in claim 1, wherein R² represents —OH.

30. A compound as defined in claim 1, or a pharmaceutically-acceptable salt thereof, for use as a pharmaceutical.

31. A pharmaceutical formulation including a compound as defined in claim 1, or a pharmaceutically-acceptable salt thereof, in admixture with a pharmaceutically acceptable adjuvant, diluent or carrier.

32.-35. (canceled)

36. The method of claim 37, wherein the disease is asthma, chronic obstructive pulmonary disease, pulmonary fibrosis, inflammatory bowel disease, irritable bowel syndrome, inflammatory pain, fever, migraine, headache, low back pain, fibromyalgia, a myofascial disorder, a viral infection, a bacterial infection, a fungal infection, dysmenorrhea, a burn, a surgical or dental procedure, a malignancy, hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, a neurodegenerative disorder, an autoimmune disease, an allergic disorder, rhinitis, an ulcer, coronary heart disease, sarcoidosis, any other disease with an inflammatory component, osteoporosis, osteoarthritis, Paget's disease or a periodontal disease.

37. A method of treatment of a disease in which inhibition of the activity of a member of the MAPEG family is desired and/or required, which method comprises administration of a therapeutically effective amount of a compound as defined in claim 1, or a pharmaceutically-acceptable salt thereof, to a patient suffering from, or susceptible to, such a condition.

38. A method as claimed in claim 37, wherein the member of the MAPEG family is microsomal prostaglandin E synthase-1, leukotriene C₄ and/or 5-lipoxygenase-activating protein.

39. A method as claimed in claim 38, wherein the member of the MAPEG family is microsomal prostaglandin E synthase-1.

40. A combination product comprising:
(A) a compound as defined in claim 1, or a pharmaceutically-acceptable salt thereof; and
(B) another therapeutic agent that is useful in the treatment of inflammation, wherein each of components (A) and (B) is formulated in admixture with a pharmaceutically-acceptable adjuvant, diluent or carrier.

41. A combination product as claimed in claim 40 which comprises a pharmaceutical formulation including a compound as defined in claim 1, or a pharmaceutically-acceptable salt thereof, another therapeutic agent that is useful in the treatment of inflammation, and a pharmaceutically-acceptable adjuvant, diluent or carrier.

42. A combination product as claimed in claim 40 which comprises a kit of parts comprising components:
(a) a pharmaceutical formulation including a compound as defined in claim 1, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

43. A process for the preparation of a compound as defined in claim 1, which comprises:
(i) reaction of a compound of formula II, 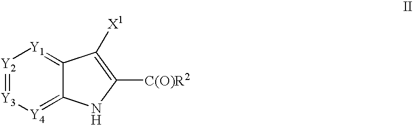
wherein X¹, R², Y₁, Y₂, Y₃ and Y₄ are as defined in claim 1, with a compound of formula III,
R¹L¹ III
wherein L¹ represents a suitable leaving group and R¹ is as defined in claim 1;
(ii) for compounds of formula I in which X¹ represents -Q-X², in which Q is a single bond or —C(O)—, reaction of a compound of formula IV, 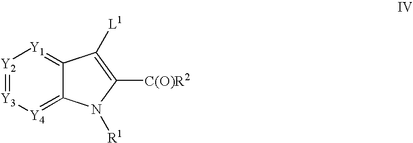
wherein R¹, R², Y₁, Y₂, Y₃ and Y₄ are as defined in claim 1 and L¹ is as defined above, with a compound of formula V,
X²-Q^{a}-L² V
wherein Q^{a} represents a single bond or —C(O)—, L² represents a suitable leaving group and
X² is as defined in claim 1;
(iii) for compounds of formula I in which X¹ represents -Q-X² and Q represents —C(O)—, reaction of a compound of formula I in which X¹ represents H with a compound of formula V in which Q^{a} represents —C(O)—;
(iv) for compounds of formula I in which X¹ represents —N(R⁸)-J-R⁹ or -Q-X² in which Q represents —O— or —S—, reaction of a compound of formula IV as defined above with a compound of formula VI,
X^{1b}H VI
in which X^{1b} represents —N(R⁸)-J-R⁹ or -Q-X² and Q represents —O— or —S— and R⁸, J, R⁹ and X² are as defined in claim 1;
(v) for compounds of formula I in which X¹ represents -Q-X² and Q represents —S—, reaction of a compound of formula I in which X¹ represents H, with a compound of formula VI in which X^{1b} represents -Q-X², Q represents —S— and X² is as defined in claim 1;
(vi) for compounds of formula I in which X¹ represents -Q-X² and Q represents —S(O)— or —S(O)₂—, oxidation of a corresponding compound of formula I in which Q represents —S—;
(vii) for compounds of formula I in which X¹ represents -Q-X², X² represents C₁₋₈ alkyl substituted by G¹, G¹ represents -A¹-R^{12a}, A¹ represents —N(R^{13a})A⁴- and A⁴ is a single bond (provided that Q represents a single bond when X² represents substituted C₁ alkyl), reaction of a compound of formula VII, 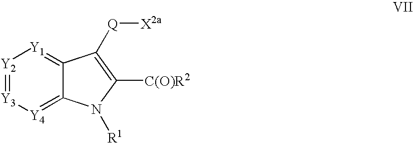
wherein X^{2a} represents a C₁₋₈ alkyl group substituted by a Z¹ group in which Z¹ represents ═O, Q is as defined in claim 1, provided that it represents a single bond when X^{2a} represents C₁ alkyl substituted by ═O, and R¹, R², Y₁, Y₂, Y₃ and Y₄ are as defined in claim 1 under reductive amination conditions in the presence of a compound of formula VIII,
R^{12a}(R^{13a})NH VIII
wherein R^{12a} and R^{13a} are as defined in claim 1;
(viia) for compounds of formula I in which X¹ represents -Q-X², Q represents a single bond, X² represents methyl substituted by G¹, G¹ represents -A¹-R^{12a}, A¹ represents —N(R^{13a})A⁴- and A⁴ is a single bond, reaction of a corresponding compound of formula I in which X¹ represents H, with a mixture of formaldehyde (or equivalent reagent) and a compound of formula VIII as defined above;
(viii) for compounds of formula I in which X¹ represents -Q-X², Q represents a single bond and X² represents optionally substituted C₂₋₈ alkenyl (in which a point of unsaturation is between the carbon atoms that are É and é to the indole ring), reaction of a corresponding compound of formula IV in which L¹ represents halo with a compound of formula IXA,
H₂C═C(H)X^{2b} IXA
or reaction of a compound of formula VII in which Q represents a single bond and X^{2a} represents —CHO with either a compound of formula IXB,
(EtO)₂P(O)CH₂X^{2b} IXB
or the like, or a compound of formula IXC,
(Ph)₃P═CHX^{2b} IXC
or the like, wherein, in each case, X^{2b} represents H, G¹ or C₁₋₆ alkyl optionally substituted with one of more substituents selected from G¹ and/or Z¹ and G¹ and Z¹ are as defined in claim 1;
(ix) for compounds of formula I in which X¹ represents -Q-X² and X² represents optionally substituted, saturated C₂₋₈ alkyl, saturated cycloalkyl, saturated heterocycloalkyl, C₂₋₈ alkenyl, cycloalkenyl or heterocycloalkenyl, reduction of a corresponding compound of formula I in which X² represents optionally substituted C₂₋₈ alkenyl, cycloalkenyl, heterocycloalkenyl, C₂₋₈ alkynyl, cycloalkynyl or heterocycloalkynyl (as appropriate);
(x) for compounds of formula I in which D represents a single bond, —C(O)—, —C(R¹⁰)(R¹¹)—, C₂₋₄ alkylene or —S(O)₂—, reaction of a compound of formula X, 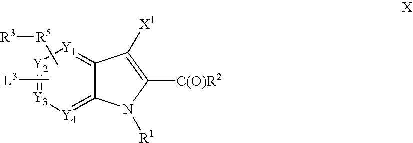
wherein L³ represents L¹ or L² as defined above, which group is attached to one or more of the carbon atoms of the pyridine ring of the pyrrolopyridine, R³-R⁵ represents whichever of the two other substituents on the pyridine ring are already present in that ring, and X¹, R¹, R², Y₁ to Y₄, R³, R⁴ and R⁵ are as defined in claim 1, with a compound of formula XI,
E-D^{a}-L⁴ XI
wherein D^{a} represents a single bond, —C(O)—, —C(R¹⁰)(R¹¹)—, C₂₋₄ alkylene or —S(O)₂—, L⁴ represents L¹ (when L³ is L²) or L² (when L³ is L¹), E, R¹⁰ and R¹¹ are as defined in claim 1 and L¹ and L² are as defined above;
(xi) for compounds of formula I in which D represents —S—, —O— or C₂₋₄ alkynylene in which the triple bond is adjacent to E, reaction of a compound of formula X as defined above in which L³ represents L² as defined above with a compound of formula XII,
E-D^{b}-H XII
wherein D^{b} represents —S—, —O— or C₂₋₄ alkynylene in which the triple bond is adjacent to E and E is as defined in claim 1;
(xii) for compounds of formula I in which D represents —S(O)— or —S(O)₂—, oxidation of a corresponding compound of formula I in which D represents —S—;
(xiii) for compounds of formula I in which D represents —O— or —S—, reaction of a compound of formula XIII, 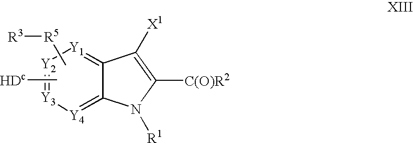
wherein the -D^{c}-H group is attached to one or more of the carbon atoms of the pyridine ring of the pyrrolopyridine, D^{c} represents —O— or —S— and X¹, R¹, R² and Y₁ to Y₄ are as defined in claim 1 and R³-R⁵ is as defined above, with a compound of formula XIV,
E-L² XIV
wherein L² is as defined above and E is as defined in claim 1;
(xiv) for compounds of formula I in which X¹ represents —N(R⁸)-J-R⁹, reaction of a compound of formula XV, 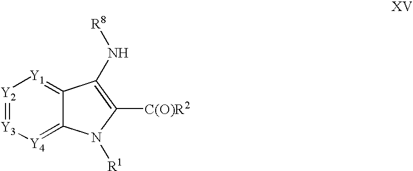
wherein R¹, R², Y₁ to Y₄ and R⁸ are as defined in claim 1, with a compound of formula XVI,
R⁹-J-L XVI
wherein J and R⁹ are as defined in claim 1 and L¹ is as defined above;
(xv) for compounds of formula I in which X¹ represents —N(R⁸)-J-R⁹, J represents a single bond and R⁹ represents a C₁₋₈ alkyl group, reduction of a corresponding compound of formula I, in which J represents —C(O)— and R⁹ represents H or a C₁₋₇ alkyl group, in the presence of a suitable reducing agent;
(xvi) for compounds of formula I in which X¹ represents halo, reaction of a compound of formula I wherein X¹ represents H, with a reagent or mixture of reagents known to be a source of halo atoms;
(xvii) for compounds of formula I in which R² represents —OR^{6a} and R^{6a} is other than H, reaction of a compound of formula XVII, 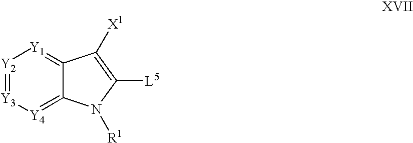
wherein L⁵ represents an appropriate alkali metal group, a —Mg-halide, a zinc-based group or a suitable leaving group, or a protected derivative thereof, and X¹, R¹ and Y₁ to Y₄ are as defined in claim 1, with a compound of formula XVIII,
L⁶C(O)OR^{6za} XVIII
wherein R^{6za} represents R^{6a} provided that it does not represent H, and L⁶ represents a suitable leaving group;
(xviii) for compounds of formula I in which R² represents —OR^{6a} and R^{6a} is H, reaction of a compound of formula XVII in which L⁵ represents either:
(I) an alkali metal; or
(II)—Mg-halide,
with carbon dioxide, followed by acidification;
(xix) for compounds of formula I in which R² represents —OR^{6a}, reaction of a corresponding compound of formula XVII in which L⁵ is a suitable leaving group with CO (or a reagent that is a suitable source of CO), in the presence of a compound of formula XIX,
R^{6a}OH XIX
wherein R^{6a} is as defined in claim 1, and an appropriate catalyst system;
(xx) for compounds of formula I in which R² represents —OR^{6a} and R^{6a} represents H, hydrolysis of a corresponding compound of formula I in which R^{6a} does not represent H;
(xxi) for compounds of formula I in which R² represents —OR^{6a} and R^{6a} does not represent H:
(A) esterification of a corresponding compound of formula I in which R^{6a} represents H; or
(B) trans-esterification of a corresponding compound of formula I in which R^{6a} does not represent H (and does not represent the same value of R^{6a} as the compound of formula I to be prepared),
in the presence of the appropriate alcohol of formula XIX as defined above but in which R^{6a} represents R^{6za} as defined above;
(xxii) for compounds of formula I in which R² represents —N(R^{6b})R⁷, reaction of a corresponding compound of formula I in which R² represents —OR^{6a} with a compound of formula XX,
HN(R^{6b}R⁷ XX
wherein R^{6b} and R⁷ are as defined in claim 1;
(xxiii) for compounds of formula I in which X¹ represents -Q-X² in which Q represents —O—, reaction of a compound of formula XXI, 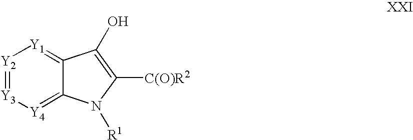
wherein R¹, R² and Y₁ to Y₄ are as defined in claim 1, with a compound of formula XXII,
X²L⁷ XXII
wherein L⁷ represents a suitable leaving group and X² is as defined in claim 1;
(xxiv) for compounds of formula I in which X¹ represents —N(R⁸)-J-R⁹, reaction of a compound of formula XXI as defined above, with a compound of formula VI in which X^{1b} represents —N(R⁸)-J-R⁹ and R⁸, R⁹ and J are as defined in claim 1;
(xxv) for compounds of formula I in which X¹ represents -Q-X², Q represents a single bond and X² represents C₁₋₈ alkyl or heterocycloalkyl substituted a to the indole ring by a G¹ substituent in which G¹ represents -A¹-R^{12a}, A¹ represents -OA⁵-, A⁵ represents a single bond and R^{12a} represents H, reaction of a corresponding compound of formula I in which X¹ represents H with a compound corresponding to a compound of formula VI, but in which X^{1b} represents -Q-X², Q represents a single bond and X² represents C₁₋₈ alkyl or heterocycloalkyl, both of which groups are substituted by a Z¹ group in which Z¹ represents ═O;
(xxvi) for compounds of formula I in which X¹ represents -Q-X², Q represents a single bond and X² represents C₂₋₈ alkyl substituted by a G¹ substituent in which G¹ represents -A¹-R^{12a}, A¹ represents —OA⁵-, A⁵ represents a single bond and R^{12a} represents H, reaction of a corresponding compound of formula I in which X² represents C₁₋₇ alkyl substituted by a Z¹ group in which Z¹ represents ═O, with the corresponding Grignard reagent derivative of a compound of formula V in which L² represents chloro, bromo or iodo, Q^{a} is a single bond and X² represents
C₁₋₇ alkyl;
(xxvii) for compounds of formula I in which X¹ represents -Q-X², Q represents a single bond, and X² represents C₁₋₈ alkyl or heterocycloalkyl, both of which are unsubstituted in the position a to the indole ring, reduction of a corresponding compound of formula I in which X² represents C₁₋₈ alkyl substituted a to the indole ring by a G¹ substituent in which G¹ represents -A¹-R^{12a}, A¹ represents —OA⁵-, A⁵ represents a single bond and R^{12a} represents H; or
(xxviii) for compounds of formula I in which X¹ represents -Q-X², Q represents a single bond and X² represents C₁₋₈ alkyl or heterocycloalkyl, neither of which are substituted by Z¹ in which Z¹ represents ═O, reduction of a corresponding compound of formula I in which X² represents C₁₋₈ alkyl or heterocycloalkyl, which groups are substituted by one or more Z¹ groups in which Z¹ represents ═O.
